(19) United States
(12) Patent Application Publication
(10) Pub. No.: US 2024/0353424 A1
(43) Pub. Date: Oct. 24, 2024

(54) BIOMARKERS TO DETECT RISK OF SINUSOIDAL OBSTRUCTION SYNDROME

(71) Applicant: MUSC Foundation for Research Development, Charleston, SC (US)

(72) Inventor: SOPHIE PACZESNY, Charleston, SC (US)

(73) Assignee: MUSC Foundation for Research Development, Charleston, SC (US)

(21) Appl. No.: 18/632,697

(22) Filed: Apr. 11, 2024

Related U.S. Application Data
(60) Provisional application No. 63/459,100, filed on Apr. 13, 2023.

(51) Int. Cl. G01N 33/68; A61K 31/198; A61K 31/7084; A61K 38/36; A61P 1/16; C07K 16/18; C07K 16/40; G01N 33/543

(52) U.S. Cl. CPC G01N 33/6869; A61K 31/198; A61K 31/7084; A61K 38/366; A61P 1/16; C07K 16/18; C07K 16/40; G01N 33/543; G01N 33/6893; G01N 2333/705; G01N 2800/085; G01N 2800/52

(57) ABSTRACT

The present disclosure provides biomarker panels for evaluating subjects at risk of sinusoidal obstruction syndrome (SOS) early after hematopoietic stem cell transplantation (HSCT). In particular, the present disclosure relates to the use of ST2. L-Ficolin, and HA for prognosing, diagnosing, and/or treating SOS.

This application claims benefit of priority to U.S. Provisional Application Ser. No. 63/459,100 filed Apr. 13, 2023, the entire contents of which are hereby incorporated by reference.

### STATEMENT OF FEDERAL FUNDING

This invention was made with government support under grant nos. R01 CA168814, P50 HD090215, and R01 HD074587 awarded by the National Institutes of Health. The government has certain rights in the invention.

### BACKGROUND

### 1. Field

The disclosure relates generally to the field of biomarkers. Specifically, it concerns methods to detect risk and prognostic biomarkers that, respectively, (i) indicate the potential for developing a medical condition in an individual who does not currently have clinically apparent disease or the medical condition, and that (ii) identify likelihood of a clinical event or progression in patients who have the medical condition of interest.

### 2. Related Art

Allogeneic hematopoietic cell transplantation (HCT) is a potentially curative therapy for blood disorders. However, the efficacy of this procedure has been impeded by early endothelial dysfunction that can lead to a severe and potentially lethal complication called sinusoidal obstruction syndrome (SOS), also known as veno-occlusive disease (VOD) (1). Despite less aggressive conditioning regimens leading to a decrease in incidence in recent years, SOS that evolves to multiorgan failure (MOF) in children has a high mortality rate (2-4). As highlighted by the Pediatric Acute Lung Injury and Sepsis Investigators (PALISI) and Pediatric Blood and Marrow Transplantation Consortium (PTCTC) Joint Working Committee consensus, there is high variability in pediatric management of SOS, which may contribute to the increased morbidity and mortality (5).

Defibrotide received Food and Drug Administration (FDA) approval for SOS treatment (6-8). The safety profile of defibrotide is excellent (9). In a randomized trial, its prophylactic administration in a pediatric population resulted in decreased SOS incidence from 20% to 12%, although the p-values were just at the limit of significance (Z test for competing risk analysis p=0.0488, and log-rank test p=0.0507) (10, 11). This was not confirmed in a trial including adults and children (10, 11). Therefore, prophylactic defibrotide for SOS is controversial and its administration to all HCT patients is not practical or cost-efficient. To fill this gap, enrichment for patients at high-risk of developing SOS is needed.

SOS diagnosis and its severity are assessed late in the course of the disease by non-specific clinical signs, and laboratory assays (ascites, weight gain, hepatomegaly, right upper quadrant pain, and bilirubin ≥2 mg/dl) (12-14). Updated scoring criteria have been proposed that allow for earlier recognition of SOS but came after the start of this prospective study (15-16).

Currently, no validated laboratory test exists to stratify patients at high-risk for developing SOS. Neither pre-transplant clinical characteristics (recipient or donor) nor transplant characteristics have proved to be reliable predictors of SOS (2, 14, 17, 18). Conforming to FDA/NIH-BEST (biomarkers, endpoints, and other tools) recommendations, risk biomarkers are defined as assays that are associated with an increased susceptibility of developing a condition in an individual who does not yet have clinical evidence of that condition (19). Across multiple studies, only a few risk biomarkers for SOS have been identified but in mostly adults and in retrospective sets which has several potential limitations that can be addressed in a multicenter prospective study. The 2014 NIH Consensus development project further provided a framework for the development of biomarkers into clinical practice with a critical step of validation in a prospective “real world” cohort (20). To establish the more granular positive predictive value (PPV) and negative predictive value (NPV), biomarkers cutpoints need to be validated in a prospective study (20, 21).

Risk biomarkers of SOS have not been verified in a prospective cohort accounting for differences between practices across institutions. The present disclosure aimed to define risk groups for SOS occurrence using three proteins: L-Ficolin, Hyaluronic Acid (HA), and Stimulation-2 (ST2). Thus, there was an unmet need for biomarkers to detect risk of SOS when biomarkers are measured at days 3 and 7 post-hematopoietic cell transplantation several days before SOS occurrence.

### SUMMARY

In certain embodiments, the present disclosure provides methods for treating and/or preventing sinusoidal obstructive syndrome (SOS) in a subject receiving hematopoietic stem cell transplantation (HCT), said the method comprising:
(a) obtaining a biological sample from a subject receiving HCT;
(b) measuring in said biological sample from the subject the expression of L-Ficolin, hyaluronic acid (HA), and IL-1RL1 (ST2) by contacting the biological sample obtained from the subject with specific binding agents that specifically bind to each of the respective the biomarkers, wherein each specific binding agent forms a complex with the respective biomarker;
(c) detecting the agent-biomarker complexes, thereby determining the biomarker expression level, wherein decreased expression level of L-ficolin, elevated expression level of HA, and/or elevated expression level of ST2 as compared to a control identifies a subject at risk of SOS; and
(d) treating said patient at risk of SOS with a therapeutic agent.

In some aspects, the therapeutic agent is defibrotide, eculizumab, narsoplimab, n-acetyl-1-cysteine (NAC), and/or recombinant human soluble thrombomodulin alpha (rhTM). In particular aspects, the therapeutic agent is defibrotide.

In certain aspects, the biological sample is blood, serum, plasma, urine, spinal fluid, saliva, lacrimal fluid, or sweat. In some aspects, the biological sample is a blood sample. In particular aspects, the biological sample is a plasma sample.

In some aspects, detecting is further defined as performing an enzyme-linked immunosorbent assay (ELISA). In certain aspects, the sample was obtained from the subject three days post-HCT. In particular aspects, the sample was obtained from the subject between 1 and 30 days post-HCT, such as between 2 and 15 (e.g., 1, 2, 3, 4, 5, 6, 7, 8, 9, 10, 11, 12, 13, 14, or 15 days post-HCT) days post-HCT, between three and seven days post-HCT, at 3 days post-HCT, or at 7 days post-HCT. In certain aspects, the subject is less than 18 years of age (e.g., 17, 16, 15, 14, 13, 12, 11, 10, 9, 8, 7, 6, 5, 4, 3, 2 or 1 years of age) less than 16 years of age, less than 5 years of age, or the subject is 3 years of age or less. In particular aspects, the control is a healthy subject.

In specific aspects, an expression level of L-ficolin between 880 to 1,320 ng/ml, expression level of HA between 160 to 240 ng/ml, and/or an expression level of ST2 between 36 to 54 ng/mL identifies a subject at risk of SOS. In specific aspects, an expression level of L-ficolin less than 1,320 ng/ml, expression level of HA greater than 160 ng/mL, and/or an expression level of ST2 greater than 36 ng/mL identifies a subject at risk of SOS. In some aspects, an expression level of L-ficolin less than 1100 ng/mL, expression level of HA greater than 200 ng/ml, and/or an expression level of ST2 greater than 45 ng/ml identifies a subject at risk of SOS. In certain aspects, decreased expression level of L-ficolin and elevated expression level of ST2 as compared to a control identifies a subject at risk of SOS. In certain aspects, decreased expression level of L-ficolin, elevated expression level of HA, and elevated expression level of ST2 as compared to a control identifies a subject at risk of SOS. In some aspects, administering defibrotide results in increased expression levels of L-ficolin, decreased expression level of HA, and/or decreased expressed level of ST2 as compared to expression levels prior to defibrotide treatment. In some aspects, the expression of VCAM or ANG2 is not determined. In other aspects, the expression of VCAM and/or ANG2 is determined.

In some aspects, defibrotide is administered for less than 21 days, less than 15 days, or less than 10 days. In certain aspects, the AUC of each of the biomarkers is greater than 0.5, 0.6, 0.7, or 0.8. In some aspects, the detection for the biomarkers has a sensitivity greater than 60%, 70%, or 80%. In certain aspects, the detection for the biomarkers has a specificity greater than 70% or 80%.

In certain aspects, the method further comprises detecting the level of bilirubin in said biological sample. In some aspects, an increased level of ST2 identifies early endothelium damage and an increased level of HA and/or a decreased level of L-Ficolin identifies liver damage in said subject. In some aspects, an expression level of L-ficolin less than 1100 ng/ml, an expression level of HA greater than 200 ng/ml, and/or an expression level of ST2 greater than 45 ng/ml indicates decreased survival as compared to median survival in a subject having SOS. In particular aspects, a positive or high score correlates with SOS and SOS correlated with lower (i.e., worst) survival.

A further embodiment provides an in vitro method for measuring the expression of the antigens L-ficolin, HA, and ST2 comprising:
(a) obtaining a sample from a subject having received a hematopoietic stem cell transplantation (HCT);
(b) contacting said sample with an anti-L-ficolin antibody, an anti-HA antibody, and an anti-ST2 antibody to form antigen-antibody complexes; and
(c) detecting the antigen-antibody complexes using detectable moieties that distinctly bind each of the antibodies, thereby measuring the expression of the antigens L-ficolin, HA, and ST2 in said sample.

In some aspects, the method does not comprise detecting VCAM or ANG2. In other aspects, the method comprises detecting VCAM or ANG2. In certain aspects, the sample is blood, serum, plasma, urine, spinal fluid, saliva, lacrimal fluid, or sweat. In certain aspects, the sample is a blood sample. In particular aspects, the sample is a plasma sample.

In some aspects, detecting is further defined as performing an enzyme-linked immunosorbent assay (ELISA). In particular aspects, the antibodies of step (a) are conjugated to a surface. In certain aspects, measuring comprises comparing the expression of each of the three antigens to the expression in a control sample. In some aspects, the control sample is isolated from a healthy subject.

In particular aspects, a decreased expression level of L-ficolin, elevated expression level of HA, and/or elevated expression level of ST2 as compared to a control identifies a subject at risk of SOS and/or decreased survival compared to median survival in a subject having SOS. In some aspects, the specificity of the assay is at least 0.7, 0.8, or 0.9. In certain aspects, the AUC of the assay is at least 0.6, 0.7, or 0.8. In certain aspects, the AUC of each of the biomarkers is greater than 0.5, 0.6, 0.7, or 0.8.

In particular aspects, the sample was obtained from the subject between 1 and 30 days post-HCT, such as between 2 and 15 (e.g., 1, 2, 3, 4, 5, 6, 7, 8, 9, 10, 11, 12, 13, 14, or 15 days post-HCT) days post-HCT, between three and seven days post-HCT, at 3 days post-HCT, or at 7 days post-HCT. In certain aspects, the subject is less than 18 years of age (e.g., 17, 16, 15, 14, 13, 12, 11, 10, 9, 8, 7, 6, 5, 4, 3, 2 or 1 years of age) less than 16 years of age, less than 5 years of age, or the subject is 3 years of age or less. In particular aspects, the control is a healthy subject. In some aspects, the subject received a peripheral blood transplant. In certain aspects, the subject received a bone marrow transplant.

In specific aspects, an expression level of L-ficolin between 880 to 1,320 ng/mL, expression level of HA between 160 to 240 ng/mL, and/or an expression level of ST2 between 36 to 54 ng/mL identifies a subject at risk of SOS. In specific aspects, an expression level of L-ficolin less than 1,320 ng/mL, expression level of HA greater than 160 ng/mL, and/or an expression level of ST2 greater than 36 ng/mL identifies a subject at risk of SOS. In some aspects, an expression level of L-ficolin less than 1100 ng/mL, expression level of HA greater than 200 ng/mL, and/or an expression level of ST2 greater than 45 ng/mL identifies a subject at risk of SOS. In certain aspects, decreased expression level of L-ficolin and elevated expression level of ST2 as compared to a control identifies a subject at risk of SOS. In certain aspects, decreased expression level of L-ficolin, elevated expression level of HA, and elevated expression level of ST2 as compared to a control identifies a subject at risk of SOS. In some aspects, administering defibrotide results in increased expression levels of L-ficolin, decreased expression level of HA, and/or decreased expressed level of ST2 as compared to expression levels prior to defibrotide treatment, an expression level of L-ficolin less than 1100 ng/mL, an expression level of HA greater than 200 ng/mL, and/or an expression level of ST2 greater than 45 ng/mL identifies a subject at risk of SOS and/or decreased survival compared to median survival in a subject having SOS.

In additional aspects, the method further comprises administering a therapeutic composition to said subject identified as having or at risk of developing SOS. In some aspects, the therapeutic composition comprises defibrotide, eculizumab, narsoplimab, n-acetyl-1-cysteine (NAC), and/or recombinant human soluble thrombomodulin alpha (rhTM). In particular aspects, the therapeutic composition comprises defibrotide.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Study design. Workflow illustrating the study population, biomarkers measurements, and timepoints.

FIG. 2: Diagram of the algorithm for risk prediction of SOS, its confusion matrix, sensitivity, specificity, PPV, and NPV.

FIG. 3A-3C: SOS cumulative incidence by day 35 stratified by high and low day 3 risk biomarkers using optimal cutpoints chosen by Youden's index. (FIG. 3A) L-Ficolin. (FIG. 3B) HA. (FIG. 3C) ST2.

FIG. 4: Cumulative incidence of SOS stratified by the three-biomarker score.

FIGS. 5A-5C: Day 100 OS stratified by high and low day 3 risk biomarkers using optimal cutpoints chosen by Youden's index. (FIG. 5A) L-Ficolin. (FIG. 5B) HA. (FIG. 5C) ST2.

FIG. 6: Kaplan-Meier estimates of overall survival (OS) stratified by the three-biomarker score.

FIG. 7: Biomarker changes post-defibrotide treatment.

FIG. 8: Kaplan-Meier estimates of overall survival (OS) on day 100 post-transplant.

FIGS. 9A-9B: (FIG. 9A) ROC curves for day+3 biomarkers. (FIG. 9B) ROC curves for day+7 biomarkers.

FIG. 10: Schematic of trial design.

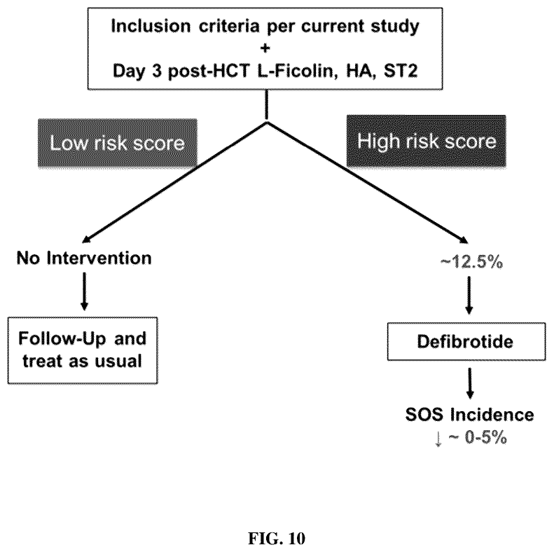

### DESCRIPTION OF ILLUSTRATIVE EMBODIMENTS

L-Ficolin, Hyaluronic Acid (HA), and Stimulation 2 (ST2) were identified and validated in three retrospective cohorts as risk biomarkers for SOS (22). An Endothelial Activation and Stress Index (EASIX) assessed at the day of transplant was significantly associated with SOS incidence (23). However, PPV and NPV values were not evaluated in these studies, therefore the biomarkers were not qualified (19, 23, 24).

In the present studies, L-Ficolin, HA, and ST2 were prospectively assessed at two early timepoints following HCT in a multicenter pediatric cohort. The biomarkers cutpoints were determined by exhaustive grid search and Youden's Index using biomarkers data from all retrospective cohorts from a previously published study (22; incorporated herein by reference). The identified biomarker cutpoints were then applied in the prospective “real world multicenter” cohort to assess their NPV and PPV as risk biomarkers for SOS for future use in biomarker-based preemptive treatment for SOS. It is important to note that this study is the first prospective biomarkers analysis reported in the field of HCT.

Specifically, the inventors report biomarkers that were tested by ELISA blind to patient groupings and associated with SOS incidence at day 3 post-HCT, and overall survival (OS) at day 100 post-HCT. Cutpoints were identified using retrospective cohorts and applied to the prospective cohort. Combination of the three biomarkers measured at day 3 post-HCT in the prospective cohort provided 80% (95% CI, 55-100%) sensitivity and 73% (95% CI, 62-83%) specificity for risk of SOS occurrence. Patients with low L-Ficolin were 9 times (95% CI 3-32) more likely to develop SOS, while patients with high HA and ST2 were 6.5 (95% CI 1.9-22.0) and 5.5 (95% CI 2.3-13.1) times more likely to develop SOS. These three markers also predicted worse day 100 OS [L-Ficolin: HR, 10.0 (95% CI 2.2-45.1), P=0.0002; HA: HR, 4.1 (95% CI 1.0-16.4), P=0.031; ST2: HR, 3.9 (95% CI 0.9-16.4), 38 P=0.04]. L-Ficolin, HA, and ST2 levels measured as early as three days post-HCT improved risk stratification for SOS occurrence and OS and may guide risk-adapted preemptive therapy. In addition, a score is provided that is validated on a prospective “real-world” cohort.

These and other aspects of the disclosure are described in detail below.

### I. Definitions

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The term “about” means, in general, within a standard deviation of the stated value as determined using a standard analytical technique for measuring the stated value. The terms can also be used by referring to plus or minus 5% of the stated value.

The phrase “effective amount” or “therapeutically effective” means a dosage of a drug or agent sufficient to produce a desired result. The desired result can be subjective or objective improvement in the recipient of the dosage, increased lung growth, increased lung repair, reduced tissue edema, increased DNA repair, decreased apoptosis, a decrease in tumor size, a decrease in the rate of growth of cancer cells, a decrease in metastasis, or any combination of the above.

“Subject” and “patient” refer to either a human or non-human, such as primates, mammals, and vertebrates. In particular embodiments, the subject is a human.

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state of a tumor or malignancy, delay or slowing of tumor growth and/or metastasis, and an increased lifespan as compared to that expected in the absence of treatment.

As used herein, the term “biomarker” refers to an indicator of, for example, a pathological state of a subject, which can be detected in a biological sample of the subject. Biomarkers include DNA-based, RNA-based and protein-based molecular markers.

As used herein, the term “diagnosis” refers to the identification or classification of a molecular or pathological state, disease or condition. For example, “diagnosis” can refer to identification of a particular type of a condition.

As used herein, the term “aiding diagnosis” refers to methods that assist in making a clinical determination regarding the presence, or nature, of a particular type of symptom or condition of a condition. For example, a method of aiding diagnosis of a condition can include measuring the expression of certain genes in a biological sample from an individual.

As used herein, the term “prognosis” is used herein to refer to the categorization of patients by degree of risk for a disease or progression of such disease. A “prognostic marker” refers to an assay that categorizes patients by degree of risk for disease occurrence or progression.

As used herein, the term “sample” refers to a composition that is obtained or derived from a subject of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized. A “tissue” or “cell sample” refers to a collection of similar cells obtained from a tissue of a subject or patient. The source of the tissue or cell sample may be blood or any blood constituents (e.g., whole blood, plasma, serum) from the subject. Samples include, without limitation, whole blood, plasma, serum, red blood cells, white blood cells (e.g., peripheral blood mononuclear cells), ductal lavage fluid, nipple aspirate, lymph (e.g., disseminated tumor cells of the lymph node), bone marrow aspirate, saliva, urine, stool (i.e., feces), sputum, bronchial lavage fluid, tears, fine needle aspirate (e.g., harvested by fine needle aspiration that is directed to a target, such as a tumor, or is random sampling of normal cells, such as periareolar), any other bodily fluid, a tissue sample (e.g., tumor tissue) such as a biopsy of a tumor (e.g., needle biopsy) or a lymph node (e.g., sentinel lymph node biopsy), and cellular extracts thereof. The tissue sample can also be primary or cultured cells or cell lines, Optionally, the tissue or cell sample is obtained from a disease tissue/organ. The tissue sample can contain compounds which are not naturally intermixed with the tissue in nature such as preservatives, anticoagulants, buffers, fixatives, nutrients, antibiotics, and the like.

As used herein, the terms “control”, “control cohort”, “reference sample”, “reference cell”. “reference tissue”, “control sample”, “control cell”, and “control tissue” refer to a sample, cell or tissue obtained from a source that is known, or believed, to not be afflicted with the disease or condition for which a method or composition of the present disclosure is being used to identify. The control can include one control or multiple controls. In one embodiment, a reference sample, reference cell, reference tissue, control sample, control cell, or control tissue is obtained from a healthy part of the body of the same subject or patient in whom a disease or condition is being identified using a composition or method of the present disclosure. In one embodiment, a reference sample, reference cell, reference tissue, control sample, control cell, or control tissue is obtained from a healthy part of the body of an individual who is not the subject or patient in whom a disease or condition is being identified using a composition or method of the invention.

The term “antibody” is used in its broadest sense and specifically covers, for example, monoclonal antibodies, polyclonal antibodies, antibodies with polyepitopic specificity, single chain antibodies, multi-specific antibodies and fragments of antibodies. Such antibodies can be chimeric, humanized, human and synthetic.

The term “determining an expression level” as used herein means the application of a gene specific reagent such as a probe, primer or antibody and/or a method to a sample, for example a sample of the subject and/or a control sample, for ascertaining or measuring quantitatively, semi-quantitatively or qualitatively the amount of a gene or genes. For example, a level of a gene can be determined by a number of methods including for example immunoassays including for example immunohistochemistry, ELISA, Western blot, immunoprecipitation and the like, where a biomarker detection agent such as an antibody for example, a labeled antibody, specifically binds the biomarker and permits for example relative or absolute ascertaining of the amount of polypeptide biomarker, hybridization.

“Elevated expression level” and “elevated levels” refer to an increased expression of a mRNA or a protein in a patient (e.g., a patient suspected of having or diagnosed as having SOS) relative to a control, such as subject or subjects who are not suffering from SOS.

### II. Biomarkers

In certain embodiments, the present disclosure is directed to a diagnostic biomarker panel comprising L-Ficolin, hyaluronic acid (HA), and IL-1RL1 (ST2) for diagnosis, prognosis, and/or treatment of sinusoidal obstructive syndrome (SOS), such as to prevent multiorgan failure (MOF) and/or increase overall survival (OS). Further, in one embodiment, a biomarker panel can be employed to provide opportunities for preemptive intervention to minimize the incidence and severity of SOS clinical symptoms, and thereby increase survival. The present disclosure further relates to the use of these biomarkers and biomarker panels for prognosing, diagnosing, and/or treating SOS in a subject that has received allogeneic hematopoietic cell transplant (HCT). The subject may have received a peripheral blood stem cell transplant or bone marrow transplant.

Aspects of the present disclosure include methods for diagnosing or monitoring the onset, progression, or regression of SOS in a subject by, for example, obtaining samples from a subject and assaying such samples for the presence of altered expression, such as decreased expression, of the biomarker L-ficolin (e.g., less than or equal to 1100 ng/ml) and increased expression of HA (e.g., greater than or equal to 200 ng/mL) and/or ST2 (e.g., greater than or equal to 45 ng/ml). An increased protein concentration compared to a control of one or more (e.g., at least 1, 2, 3, 4, or 5) of HA and/or ST2 and/or decreased protein concentration of L-ficolin is indicative/predictive that a subject that has, is suspected of having, or is at risk of developing SOS.

In some embodiments, the concentration of the biomarker being analyzed is increased as compared to the concentration of that biomarker in a control. For example, the concentration of the biomarker being analyzed can be at least 1.5, 2, 3, 4, 5, 6, 7, 8, 9, 10, 20, 25, 50, 75, or 100 times higher, or at least 10%, 20%, 30%, 40%, 50%, 60%, 70%, 80%, 90%, 100%, 200%, 300%, 400%, 500%, 600%, 700%, 800%, 900%, 1,000%, 1,500%, 2,000%, 2,500%, 3,000%, 3,500%, 4,000%, 4,500%, or 5,000% higher, than the concentration of that biomarker in a control.

In another aspect, the present disclosure is directed to a method of diagnosing or of aiding diagnosis of SOS in a subject receiving hematopoietic cell transplantation (HCT). The method comprises: measuring in a biological sample from the subject the expression of a biomarker panel comprising L-ficolin, ST2, and/or HA, by contacting the biological sample obtained from the subject with at least a first agent that specifically binds to L-ficolin, at least a second agent that specifically binds to ST2, at least a third agent that specifically binds to HA, wherein each specific binding agent forms a complex with the biomarker; and detecting the agent-biomarker complex, thereby determining the biomarker expression level; wherein an elevated biomarker expression level of HA and ST2 and decreased expression levels of L-ficolin compared to biomarker expression obtained from a biological sample obtained from a control is indicative of SOS.

In another aspect, the present disclosure is directed to a method of prognosing or of aiding prognosis of SOS in a subject receiving hematopoietic cell transplantation (HCT), such as a PB or BM transplant. The method comprises: measuring in a biological sample from the subject the expression of a biomarker panel comprising L-ficolin, ST2, and/or HA by contacting the biological sample obtained from the subject with at least a first agent that specifically binds to L-ficolin, at least a second agent that specifically binds to ST2, at least a third agent that specifically binds to HA, wherein each specific binding agent forms a complex with the biomarker; and detecting the agent-biomarker complex, thereby determining the biomarker expression level; wherein an decreased expression level of the biomarker L-ficolin (e.g., less than or equal to 1100 ng/ml) and increased expression of HA (e.g., greater than or equal to 200 ng/ml) and/or ST2 (e.g., greater than or equal to 45 ng/mL) in the biological sample obtained from the subject as compared a biological sample obtained from a control is indicative of a prognosis for a subject having SOS.

In one embodiment, the present disclosure is directed to a method of prognosing or of aiding in the prognosis of SOS in a subject receiving hematopoietic cell transplantation (HCT). The method comprises obtaining a biological sample from the subject; measuring in a biological sample from the subject the expression of a biomarker panel comprising L-ficolin, HA, and ST2 and determining the biomarker expression level; wherein a decreased expression of the biomarker L-ficolin (e.g., less than or equal to 1100 ng/ml) and increased expression of HA (e.g., greater than or equal to 200 ng/ml) and/or ST2 (e.g., greater than or equal to 45 ng/mL) compared to biomarker expression obtained from a biological sample obtained from a control is indicative of a prognosis for shortened survival compared to median survival in a subject having SOS, and wherein a increased expression level of L-ficolin (e.g., greater than 1100 ng/ml) and decreased expression level of HA (e.g., less than 200 ng/ml) and ST2 (e.g., less than 45 ng/ml) compared to biomarker expression obtained from a biological sample obtained from a control is indicative of a prognosis for increased survival compared to median survival in a subject having SOS.

The specific binding agent can be selected from a nucleic acid, an antibody, a receptor, and a lectin. The sample can be selected from tissue, whole blood, plasma and serum. The specific binding agent-biomarker complex can be detected using methods known to those skilled in the art such as, for example, microarray analysis, immunoassay, immunohistochemistry, and mass spectrometry. Representative immunoassays include Western blot analysis and ELISA. It has been advantageously found that the biomarker panels used in the methods of the present disclosure can be used for prognosing SOS early after HCT. Particularly, in some embodiments, the methods can be used to prognose SOS as early as 2 days from HCT, and in some embodiments, as early as 2 days from HCT, or as early as 3, 4, 5, 6, 7, 8, 9, 10, 11, 12, 13, 14, or 15 days from HCT.

The biological sample used in the methods of the present disclosure can be obtained using certain methods known to those skilled in the art. Biological samples may be obtained from vertebrate animals, and in particular, mammals. In certain instances, a biological sample is tissue, whole blood, plasma, or serum. By screening such body samples, a prognosis or diagnosis can be achieved for SOS.

As used in the various methods of the present disclosure, the terms “control”. “control value”, “reference” and “reference value” refer to an expression level value obtained from control sample”, “control cell”, and “control tissue” “reference sample”. “reference cell”, and “reference tissue” obtained from a source that is known, or believed, to not be afflicted with the condition for which a method or composition is being used to identify. It is to be understood that the control need not be obtained at the same time as the biological sample of the subject is obtained. Thus, a control value for an expression level can be determined and used for comparison of the expression level for the biological sample of the subject or the biological samples of multiple subjects.

### A. Detection Methods

The level of expression of the biomarker panel may be measured by ELISA, western blotting, mass spectrometry, a capillary immune-detection method, isoelectric focusing, an immune precipitation method or immunohistochemistry. In particular embodiments, the present methods concern performing one or more ELISA assays for detecting the expression of one or more biomarkers.

An enzyme-linked immunosorbent assay, or ELISA, may be used to measure the differential expression of a plurality of biomarkers. There are many variations of an ELISA assay. ELISA tests may be formatted for direct, indirect, competitive, or sandwich detection of the analyte. All are based on the immobilization of an antigen or antibody on a solid surface, generally a microtiter plate. The original ELISA method comprises preparing a sample containing the biomarker proteins of interest, coating the wells of a microtiter plate with the sample, incubating each well with a primary antibody that recognizes a specific antigen, washing away the unbound antibody, and then detecting the antibody-antigen complexes. The antibody-antibody complexes may be detected directly. The primary antibodies are conjugated to a detection system, such as an enzyme that produces a detectable product. The antibody-antibody complexes may be detected indirectly. For example, the primary antibody is detected by a secondary antibody that is conjugated to a detection system, as described above. The microtiter plate is then scanned and the raw intensity data may be converted into expression values using means known in the art. Single- and Multi-probe kits are available from commercial suppliers, e.g., Meso Scale Discovery (MSD).

In one ELISA method, a first, or capture, binding agent, such as an antibody that specifically binds the biomarker of interest, is immobilized on a suitable solid phase substrate or carrier. The test biological sample is then contacted with the capture antibody and incubated for a desired period of time. After washing to remove unbound material, a second, detection, antibody that binds to a different, non-overlapping, epitope on the biomarker is then used to detect binding of the polypeptide biomarker to the capture antibody. The detection antibody is preferably conjugated, either directly or indirectly, to a detectable moiety. Examples of detectable moieties that can be employed in such methods include, but are not limited to, cheminescent and luminescent agents; fluorophores such as fluorescein, rhodamine and eosin; radioisotopes; colorimetric agents; and enzyme-substrate labels, such as biotin.

The antibodies may be attached to imaging agents of use for imaging and diagnosis of various diseased organs, tissues or cell types. The antibody may be labeled or conjugated with a fluorophore or radiotracer for use as an imaging agent. Many appropriate imaging agents are known in the art, as are methods for their attachment to proteins or peptides using metal chelate complexes, radioisotopes, fluorescent markers, or enzymes whose presence can be detected using a colorimetric markers (such as, but not limited to, urease, alkaline phosphatase, (horseradish) hydrogen peroxidase and glucose oxidase). In some embodiments, the imaging conjugate will also be dual labeled with a radio-isotope in order to combine imaging through nuclear approaches and be made into a unique cyclic structure and optimized for binding affinity and pharmacokinetics. Such agents can be administered by any number of methods known to those of ordinary skill in the art including, but not limited to, oral administration, inhalation, subcutaneous (sub-q), intravenous (I.V.), intraperitoneal (I.P.), intramuscular (I.M.), or intrathecal injection, or as described in greater detail below.

In some aspects, the imaging agent is a chromophore, such as a fluorophore. Exemplary fluorophores suitable for use with the present disclosure includes rhodamine, rhodol, fluorescein, thiofluorescein, aminofluorescein, carboxyfluorescein, chlorofluorescein, methylfluorescein, sulfofiuorescein, aminorhodol, carboxyrhodol, chlororhodol, methylrhodol, sulforhodol; aminorhodamine, carboxyrhodamine, chlororhodamine, methylrhodamine, sulforhodamine, and thiorhodamine; cyanine, indocarbocyanine, oxacarbocyanine, thiacarbocyanine, merocyanine, cyanine 2, cyanine 3, cyanine 3.5, cyanine 5, cyanine 5.5, cyanine 7, oxadiazole derivatives, pyridyloxazole, nitrobenzoxadiazole, benzoxadiazole, pyren derivatives, cascade blue, oxazine derivatives, Nile red, Nile blue, cresyl violet, oxazine 170, acridine derivatives, pro flavin, acridine orange, acridine yellow, arylmethine derivatives, auramine, crystal violet, malachite green, tetrapyrrole derivatives, porphin, phtalocyanine and bilirubin; 1-dimethylaminonaphthyl-5-sulfonate, 1-anilino-8-naphthalene sulfonate, 2-p-touidinyl-6-naphthalene sulfonate, 3-phenyl-7-isocyanatocoumarin, N-(p-(2-benzoxazolyl)phenyl) maleimide, stilbenes, pyrenes, 6-FAM (Fluorescein), 6-FAM (NHS Ester), Fluorescein dT, HEX, JOE (NHS Ester), MAX, TET, ROX, TAMRA, TARMA™ (NHS Ester), TEX 615, ATTO™ 488, ATTO™ 532, ATTO™ 550, ATTO™ 565, ATTO™ RholOl, ATTO™ 590, ATTO™ 633, ATTO™ 647N, TYE™ 563, TYE™ 665, and TYE™ 705. In particular aspects, the chromophore is TAMRA.

The detectable moiety may include, but is not limited to fluorodeoxyglucose (FDG); 2′-fluoro-2′deoxy-1beta-D-arabionofuranosyl-5-ethyl-uracil (FEAU); 5-[¹²³I]-2′-fluoro-5-iodo-1β-D-arabinofuranosyl-uracil; 5-[¹²⁴I]-2′-fluoro-5-iodo-1β-D-arabinofuranosyl-uracil; 5-[¹³¹I]-2′-fluoro-5-iodo-1β-D-arabinofuranosyl-uracil, 5-[¹⁸F]-2′-fluoro-5-fluoro-1-B-D-arabinofuranosyl-uracil; 2-[¹¹I]- and 5-([¹¹C]-methyl)-2′-fluoro-5-methyl-1-β-D-arabinofuranosyl-uracil; 2-[¹¹C]-2′-fluoro-5-ethyl-1-β-D-arabinofuranosyl-uracil; 5-([¹¹C]-ethyl)-2′-fluoro-5-ethyl-1-β-D-arabinofuranosyl-uracil; 5-(2-[¹⁸F]-ethyl)-2′-fluoro-5-(2-fluoro-ethyl)-1-β-D-arabinofuranosyl-uracil, 5-[¹²³I]-2′-fluoro-5-iodovinyl-1-β-D-arabinofuranosyl-uracil; 5-[¹²⁴I]-2′-fluoro-5-iodovinyl-1-β-D-arabinofuranosyl-uracil; 5-[¹³¹I]-2′-fluoro-5-iodovinyl-1-β-D-arabinofuranosyl-uracil; 5-[¹²³I]-2′-fluoro-5-iodo-1-β-D-ribofuranosyl-uracil; 5-[¹²⁴I]-2′-fluoro-5-iodo-1-β-D-ribofuranosyl-uracil; 5-[¹³¹I]-2′-fluoro-5-iodo-1-β-D-ribofuranosyl-uracil; 5-[¹²³I]-2′-fluoro-5-iodovinyl-1-β-D-ribofuranosyl-uracil; 5-[¹²⁴I]-2′-fluoro-5-iodovinyl-1-β-D-ribofuranosyl-uracil; 5-[¹³¹I]-2′-fluoro-5-iodovinyl-1-β-D-ribofuranosyl-uracil; or 9-4-[¹⁸F]fluoro-3-(hydroxymethyl)butyl]guanine.

In some aspects, the imaging agent is a radionuclide. Suitable radionuclide labels are Tc, In, Ga, Cu, F, Lu, Y, Bi, Ac, and other radionuclide isotopes. Particularly, the radionuclide is selected from the group comprising ¹¹¹In, ^{99m}Tc, ^{94m}Tc, ⁶⁷Ga, ⁶⁶Ga, ⁶⁸Ga, ⁵²Fc, ⁶⁹Er, ⁷²As, ⁹⁷Ru, ²⁰³Pb, ⁶²Cu, ⁶⁴Cu, ⁶⁷Cu, ¹⁸⁶Re, ¹⁸⁸Re, ⁸⁶Y, ⁹⁰Y, ⁵¹Cr, ^{52m}Mn, ¹⁵⁷Gd, ¹⁷⁷Lu, ¹⁶¹Th, ¹⁶⁹Yb, ¹⁷⁵Yb, ¹⁰⁵Rh, ¹⁶⁶Dy, ¹⁶⁶Ho, ¹⁵³Sm, ¹⁴⁹Pm, ¹⁵¹Pm, ¹⁷²Tm, ¹²¹Sn, ^{177m}Sn, ²¹³Bi, ¹⁴²Pr, ¹⁴³Pr, ¹⁹⁸Au, ¹⁹⁹Au, ¹⁸F, ¹²³I, ¹²⁴I, ¹³¹I, ⁷⁵Br, ⁷⁶Br, ⁷⁷Br, and ⁸²Br, amongst others. These radionuclides are cationic and can be complexed with the chelator through the chelating group of the conjugate to form labeled compositions.

Methods of detecting and/or for quantifying a detectable label or signal generating material depend on the nature of the label. The products of reactions catalyzed by appropriate enzymes can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers. Any of the methods for detection can be performed in any format that allows for any suitable preparation, processing, and analysis of the reactions. This can be, for example, in multi-well assay plates (e.g., 96 wells or 386 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label. Imaging may be by optical imaging, ultrasound, PET, SPECT, MRI, or phototherapy.

In some aspects, the one or more assays may be sandwich ELISA assays. The three biomarkers may be detected by three separate ELISA assays, such as on three separate plates or slides for each biomarker or one plate or slide with separate wells for each biomarker.

In certain embodiments, the antigen-specific antibodies may be immobilized on a carrier or support (e.g., a bead, a magnetic particle, a latex particle, a microtiter plate well, a cuvette, or other reaction vessel). Examples of suitable carrier or support materials include agarose, cellulose, nitrocellulose, dextran, Sephadex®, Sepharose®, liposomes, carboxymethyl cellulose, polyacrylamides, polystyrene, gabbros, filter paper, magnetite, ion-exchange resin, plastic film, plastic tube, glass, polyamine-methyl vinyl-ether-maleic acid copolymer, amino acid copolymer, ethylene-maleic acid copolymer, nylon, silk, and the like. Binding agents may be indirectly immobilized using second binding agents specific for the first binding agents (e.g., mouse antibodies specific for the protein markers may be immobilized using sheep anti-mouse IgG Fc fragment specific antibody coated on the carrier or support).

In other aspects, the three biomarkers may be detected by a multiplex ELISA to detect two or three of the biomarkers simultaneously. For example, the multiplex ELISA may comprise an antibody array with capture antibodies spotted in subarrays on which the sample is incubated, non-specific proteins are washed off, and the array is incubated with a cocktail of biotinylated detection antibodies followed by a streptavidin-conjugated fluorophore which is visualized by a fluorescence laser scanner (e.g., Quantibody Multiplex ELISA Array, RayBiotech).

The presence of several different biomarkers in a test sample can be detected simultaneously using a multiplex assay, such as a multiplex ELISA. Multiplex assays offer the advantages of high throughput, a small volume of sample being required, and the ability to detect different proteins across a board dynamic range of concentrations. In certain embodiments, such methods employ an array, wherein multiple binding agents (for example, capture antibodies) specific for multiple biomarkers are immobilized on a substrate, such as a membrane, with each capture antibody being positioned at a specific, pre-determined, location on the substrate. Methods for performing assays employing such arrays include those described, for example, in U.S. Patent Publication Nos. US2010/0093557A1 and US2010/0190656A1, the disclosures of which are hereby specifically incorporated by reference.

Multiplex arrays in several different formats based on the utilization of, for example, flow cytometry, chemiluminescence or electron-chemiluminesence technology, are well known in the art. Flow cytometric multiplex arrays, also known as bead-based multiplex arrays, include the Cytometric Bead Array (CBA) system from BD Biosciences (Bedford, Mass.) and multi-analyte profiling (xMAP®) technology from Luminex Corp. (Austin, Tex.), both of which employ bead sets which are distinguishable by flow cytometry. Each bead set is coated with a specific capture antibody. Fluorescence or streptavidin-labeled detection antibodies bind to specific capture antibody-biomarker complexes formed on the bead set. Multiple biomarkers can be recognized and measured by differences in the bead sets, with chromogenic or fluorogenic emissions being detected using flow cytometric analysis.

In some aspects, the multiplex array may comprise a Proximity Extension Assay (PEA), such as the Olink® Explore platform and Olink® Target 96 and Target 48 panels. It can enable high-throughput, multiplex immunoassays of proteins using minimal volumes of serum, plasma, or almost any other type of biological sample. Each of the oligonucleotide antibody-pairs can contain unique DNA sequences allowing hybridization only to each other. Subsequent proximity extension will create unique DNA reporter sequences which are amplified by real-time PCR.

In an alternative format, a multiplex ELISA from Quansys Biosciences (Logan, UT) coats multiple specific capture antibodies at multiple spots (one antibody at one spot) in the same well on a 96-well microtiter plate. Chemiluminescence technology is then used to detect multiple biomarkers at the corresponding spots on the plate.

An antibody microarray may also be used to measure the differential expression of a plurality of biomarkers. For this, a plurality of antibodies is arrayed and covalently attached to the surface of the microarray or biochip. A protein extract containing the biomarker proteins of interest is generally labeled with a fluorescent dye or biotin. The labeled biomarker proteins are incubated with the antibody microarray. After washing to remove the unbound proteins, the microarray is scanned. The raw fluorescent intensity data may be converted into expression values using means known in the art.

### B. Methods of Diagnosis and Treatment

In one embodiment, the present disclosure is directed to a method of prognosing or of aiding in the prognosis of sinusoidal obstructive syndrome (SOS) in a subject receiving hematopoietic stem cell transplantation (HSCT). The method comprises: obtaining a biological sample from the subject; measuring in a biological sample from the subject, the expression of at least one biomarker selected from the group consisting of ST2, L-Ficolin, and HA by contacting the biological sample obtained from the subject with a specific binding agent that specifically binds to the biomarker, wherein the specific binding agent forms a complex with the biomarker; and detecting the agent-biomarker complex, thereby determining the biomarker expression level; wherein a decreased biomarker expression level of L-ficolin (e.g., less than or equal to 1100 ng/mL) and increased expression level of ST2 (e.g., greater than or equal to 200 ng/ml) and HA (e.g., greater than or equal to 45 ng/mL) as compared to biomarker expression obtained from a biological sample obtained from a control is indicative of a prognosis for shortened survival compared to median survival in a subject having SOS, and wherein an increased level of L-Ficolin and reduced biomarker expression level of HA and ST2 compared to biomarker expression obtained from a biological sample obtained from a control is indicative of a prognosis for increased survival compared to median survival in a subject having SOS.

In another embodiment, the present disclosure is directed to a method for diagnosing SOS in a subject, particularly a subject receiving hematopoietic stem cell transplantation (HSCT). The method comprises: measuring in a biological sample from the subject the expression of at least one biomarker selected from the group consisting of ST2, L-Ficolin, and HA, by contacting the biological sample obtained from the subject with a specific binding agent that specifically binds to the biomarker, wherein the specific binding agent forms a complex with the biomarker; and detecting the agent-biomarker complex, thereby determining the biomarker expression level; wherein decreased expression level of L-ficolin, elevated expression level of HA, and/or elevated expression level of ST2 compared to biomarker expression obtained from a biological sample obtained from a control is indicative of SOS.

It has been advantageously found that the biomarker panels used in the methods of the present disclosure can be used for diagnosing SOS early after HSCT. Particularly, in some embodiments, the methods can be used to diagnose SOS the same day as HSCT. In other embodiments, diagnosis can be made one week, two weeks, or three weeks from HSCT. Accordingly, the methods of diagnosing SOS can include obtaining the biological sample at day 0 from HSCT, including obtaining the sample from day 0 to day 7 from HSCT, including obtaining the sample from day 0 to day 14 from HSCT, and including obtaining the sample from day 0 to day 21 from HSCT.

The biological sample used in the methods of the present disclosure can be obtained using certain methods known to those skilled in the art. Biological samples may be obtained from vertebrate animals, and in particular, mammals. In certain instances, a biological sample is whole blood, plasma, or scrum. By screening such body samples, a prognosis or diagnosis can be achieved for SOS.

The methods disclosed herein enable the assessment of whether or not a subject having, suspected of having or at risk of developing SOS is likely to respond (e.g., likely to have greater improvement in disease as evidenced by reduced disease severity and/or disease remission/resolution) to a treatment. A subject having, suspected of having or at risk of developing SOS who is likely to respond to a therapeutic agent can be administered said therapeutic agent, such as defibrotide. The methods can be applied to individuals at risk of developing SOS including those who (i) have undergone a transplant (e.g., a hematopoietic stem cell transplant) but have not developed SOS, or (ii) are preparing for receipt of a transplant (e.g., a hematopoietic stem cell transplant).

Examples of therapeutic agents that can be used for treatment include defibrotide and/or a TA-TMA treatment that targets a pathway involved in SOS, the complement, such as Eculizumab or Narsoplimab. Additional agents include n-acetyl-1-cysteine (NAC) for early liver toxicity and recombinant Human Soluble Thrombomodulin Alpha (rhTM).

### III. Kits

Also within the scope of the invention are kits for performing ELISA assays on plasma samples to detect SOS. An example of such a kit may include a set of antibodies specific for the present biomarkers. The kit may further comprise instructions for use of the antibodies for performing an ELISA assay to identify altered expression of the biomarkers in the plasma samples. The kit may further comprise instructions for diagnostic purposes, indicating that elevated expression of the biomarker panel from a patient indicates an increased risk for SOS. The kit may further comprise instructions that indicate that altered expression of the biomarkers panel in a plasma indicates that a patient should be sent for further diagnostic testing and/or treated with therapeutics for SOS.

In some embodiments, a kit may further comprise detection reagents, such as streptavidin-conjugated antibodies. In some embodiments, a kit may further comprise reagents and buffers including but not limited to wash buffers. In some embodiments, a kit may further comprise mounting media and/or one or more control ELISA plates.

### IV. Examples

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1-Prospective Assessment of Risk Biomarkers of Sinusoidal Obstruction Syndrome After Hematopoietic Cell Transplantation

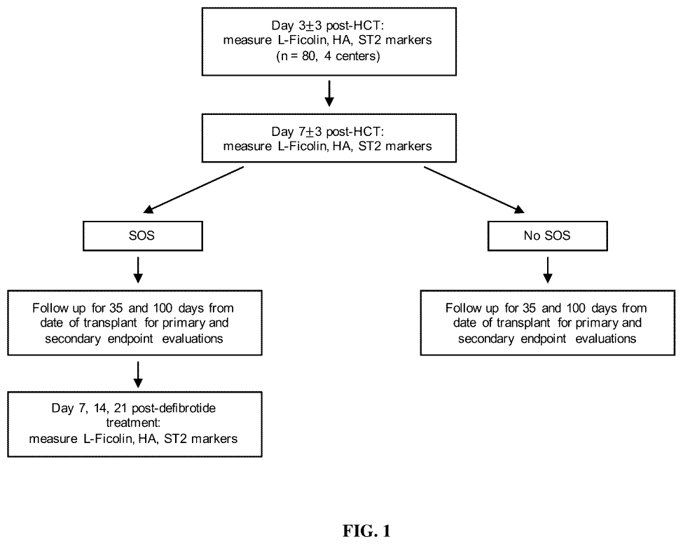

Participant demographics. 80 pediatric patients were prospectively accrued from 4 US academic health centers: Indiana University School of Medicine, Texas Children's Hospital, University of Michigan, and Children's National Medical Center. This trial was registered at Clinical Trials as NCT03132337 with detailed inclusion and exclusion criteria. The study design is summarized in FIG. 1. To allow for better accrual in mid-size pediatric centers, criteria were more permissive than the published pediatric defibrotide prophylaxis study (10). A sample size of 80 was estimated based on the placebo group in the aforementioned European study. Demographics is displayed in Table 1.

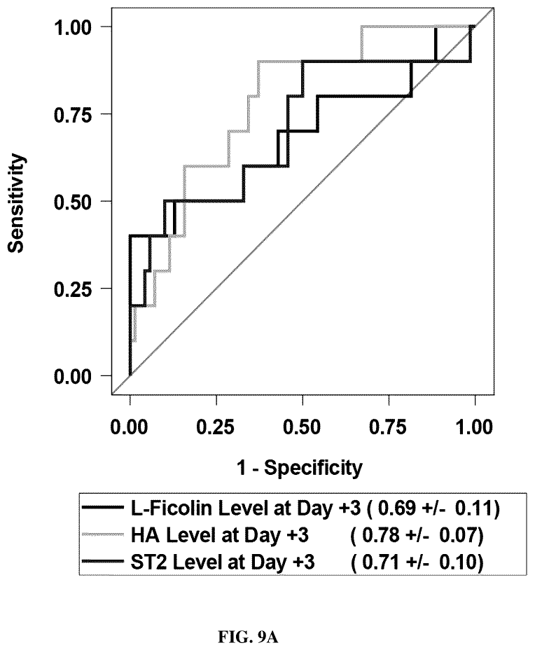

A total of 10/80 patients (12.5%) developed SOS. The median day post-HCT of SOS onset was 19 (range 9-34, Table 2). Using category of age, younger children of less than 3 years were overrepresented in the SOS group (P=0.016, Table 1). There was overrepresentation of cord transplants (P=0.038) among patients with SOS. GVHD prophylaxis with anti-thymocyte globulin (ATG) (P=0.002) was greater among patients with SOS compared to those without. Use of serotherapy with ATG or alemtuzumab was associated with cord transplant. Although seventy percent of patients who developed SOS received busulfan in the targeted exposure range (AUC>900), no significant difference was reached for conditioning regimen. Day 100 OS for the whole cohort was 91% (95% CI: 82-96%, FIG. 8). Causes of death of SOS patients was MOF in all cases. In non-SOS patients, one patient developed IPS, and one had relapse of the underlying malignant disease (Table 2 footnote).

Biomarkers for SOS risk. Levels of 3 biomarkers (L-Ficolin, HA, and ST2) previously identified in a proteomic study (22) were measured in plasma from 80 patients at two timepoints (days 3 and 7 post-HCT). Of note, ST2 test was selected over VCAM due to unpublished preliminary pre-clinical and clinical data showing its early relevance in endothelial damage following HCT. VCAM was not selected due to the poor performance of the ELISA test with wide intra and inter-assays variabilities which was not seen with L-Ficolin, HA, and ST2 ELISA tests. Descriptive statistics and correlation among the biomarkers are shown in Tables 8 and 9. L-Ficolin levels were significantly and inversely correlated with HA (P=0.0025 at day 3 and 0.0001 at day 7) but not ST2, and HA levels were positively correlated with ST2 (P<0.0001 at day 3 and <0.0001 at day 7), suggesting that the biomarkers represent different pathogenesis pathways. Expectedly, each marker was highly correlated between their values at day 3 and at day 7 suggesting that one measurement might suffice.

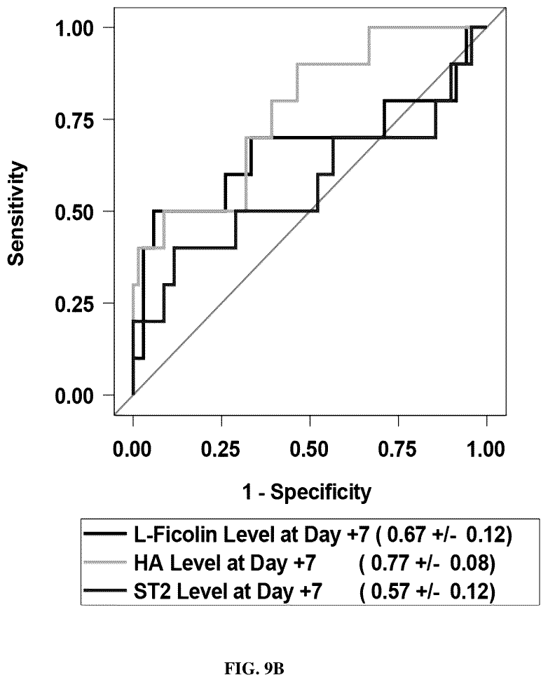

The AUCs of the ROCs were next calculated at day 3 and day 7 post-HCT for each marker (FIG. 9). The 3 biomarkers at day 3 showed AUCs between 0.69 and 0.78 and slightly lower at day 7 between 0.57 and 0.77. It was therefore hypothesized that the three biomarkers for risk evaluation of developing SOS would be most informative at day 3 post-HCT and is also concurrent with the peak of endothelial damage.

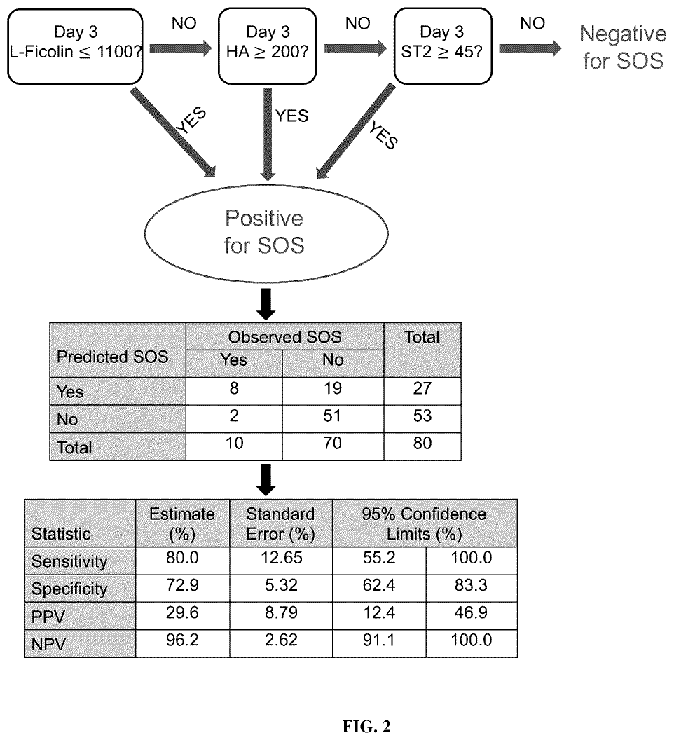

Cutpoints were determined by exhaustive grid search and Youden's index based on previous retrospective study. The retrospective included biomarkers data from three cohorts of matched cases (n=34) and controls (n=31). Cutpoints for L-Ficolin, HA, and ST2 were 1100, 200, and 45 ng/ml respectively. Based on these cutpoints, an algorithm was developed for risk prediction of SOS interrogating L-Ficolin first, and advancing to HA second, and ST2 last. Using this retrospective data, sensitivity, specificity, PPV, and NPV based on the algorithm were 64.7%, 74.2%, 73.3%, and 65.7 respectively (Table 10). When the algorithm was applied to the prospective cohort, sensitivity, specificity, PPV and NPV for the development of SOS were 80%, 72.9%, 29.6%, and 96.2%, respectively (FIG. 2). Regarding the sensitivity, specificity, PPV and NPV of individual biomarker using each cutpoint for the development of SOS in prospective cohort, there were at least 40%, 82.9%, 33.3%, and 91.3% of sensitivity, specificity, PPV and NPV, respectively (Table 11). Of note, this algorithm showed the best performance as compared to two other statistical analyses using (i) highest sensitivity for at least 50% specificity, and (ii) regression tree. Importantly, both false positive rate, and false negative rates using the algorithm were low at 27.1% and 20.0%, respectively.

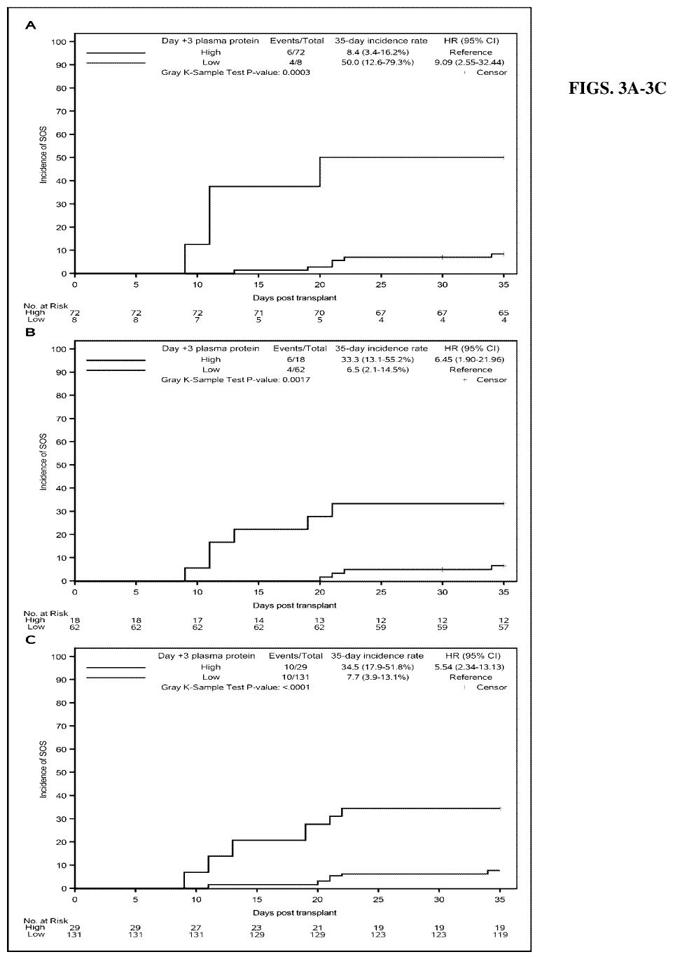

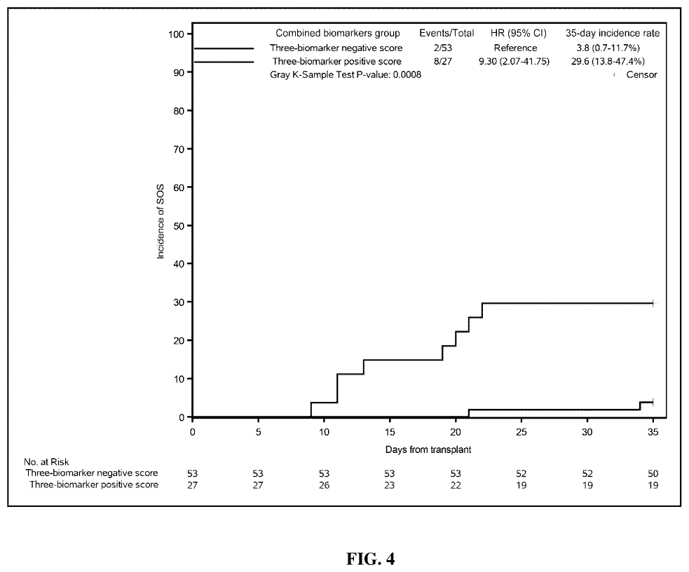

For clinical applicability, biomarkers were then dichotomized into high- and low-risk groups based on cutpoints determined above. Cumulative incidence curves of SOS stratified by day 3 biomarkers were generated individually. Low levels of L-Ficolin (<1100), and high levels of HA (>200) and ST2 (>45) were associated with a greater cumulative incidence of SOS. As compared with patients with high L-Ficolin values, patients with low L-Ficolin values were 9.1 times as likely to develop SOS (95% CI 2.6-32.4, P=0.0003). High HA and high ST2 were also associated with SOS [HA: hazard ratio (HR), 6.5; 95% CI 1.9-22.0, P=0.0017; ST2: HR, 5.5; 95% CI 2.3-13.1, P<0.0001] (FIG. 3). To investigate the effect of the combination of the three biomarkers on SOS risk, the three day 3 biomarkers dichotomized as above were incorporated into a Cox proportional hazards regression model to create a three-biomarker score. Low L-Ficolin had a beta estimate of 2.47 with 95% CI of 0.85-4.10. High HA and ST2 had a beta estimate of 1.12 with 95% CI of −0.24-2.48 and 2.11 with 95% CI of 0.56-3.67, respectively (Table 3). Combined biomarkers were divided into two groups: three-biomarker positive score and three-biomarker negative score, based on the score in Cox proportional hazards regression analysis. Compared with patients with three-biomarker negative score, patients with three-biomarker positive score were 9.3 times more likely to develop SOS (95% CI 2.1-41.8, P=0.0008) (FIG. 4).

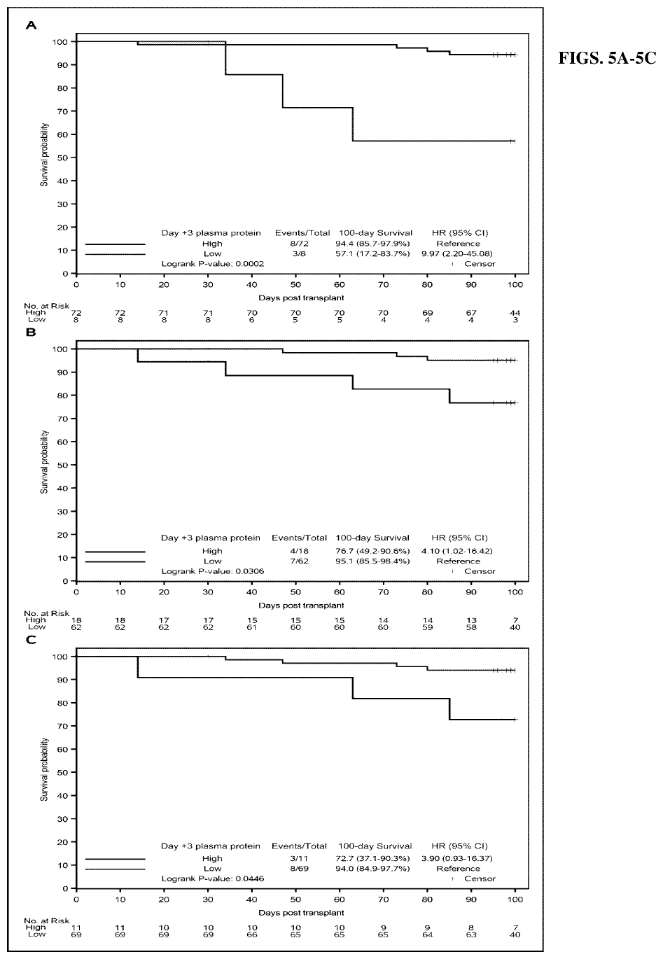

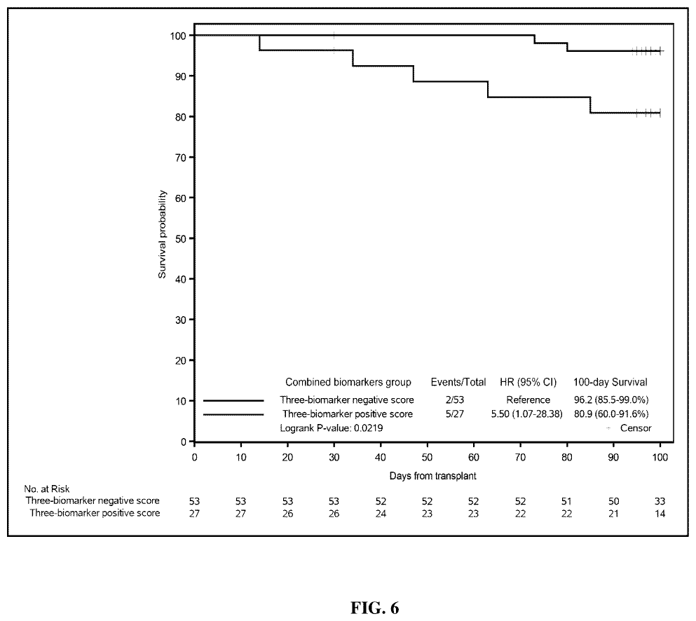

Biomarkers for prognostic of day 100 OS. Next, the ability of L-Ficolin, HA, and ST2 on day 3 to predict OS by day 100 post-HCT was examined using the same cutpoints as for SOS risk. Patients with low L-Ficolin, high HA, and high ST2 on day 3, had a lower OS at day 100 [L-Ficolin: HR, 10.0 (95% CI 2.2-45.1), P=0.0002; HA: HR, 4.1 (95% CI 1.0-16.4), P=0.031; ST2: HR, 3.9 (95% CI 0.9-16.4), P=0.045] (FIG. 5). For the combination of three day 3 biomarkers, patients with three-biomarker positive score had a lower OS at day 100 with HR of 5.5 (95% CI 1.1-28.4, P-0.0219) than those with three-biomarker negative score (FIG. 6). Therefore, even as early as day 3 post-HCT, L-Ficolin, HA and ST2 predicted worse survival within 100 days after transplant.

Associations of biomarkers with potential confounding and multivariable analysis. Currently, the diagnosis of SOS includes clinical characteristics and measurement of bilirubin) (12, 13). Thus, biomarkers levels on day 3 were compared with maximum total serum bilirubin, and day 3 total serum bilirubin using Pearson's coefficients. Even though both HA and ST2 at day 3 were correlated with maximum total serum bilirubin, only ST2 was correlated with total bilirubin measured at day 3 (Tables 12 and 13). This may suggest that early measurement of ST2 may represent an early liver damage marker. Similarly, when other liver function parameters [alkaline phosphatase (ALP), aspartate aminotransferase (AST), and alanine aminotransferase (ALT)] were measured and correlated to the day 3 biomarkers, only ST2 was slightly associated with ALP while there was no correlation between any biomarker and AST, ALT on day 3 (Tables 14-16), indicating that ST2 might correlate with early bile ducts obstruction.

Considering that several other potential complications could occur during the first 35 days such as cytokine storm (CRS) even if subclinical, thrombotic microangiopathy (TMA) which is typically accompanied by endothelial damage like SOS, idiopathic pneumonia syndrome (IPS), graft-versus-host disease (GVHD), and infections, the correlation of the day 3 SOS biomarkers were examined with these 4 potential early complications. Demographics for these other complications are shown in Table 17.

As there was no observation of any clinical CRS, two key inflammatory cytokines post-HCT [Interleukin-6 (IL-6) and Tumor Necrosis Factor Receptor-1 (TNFR1)] were used as surrogates for subclinical CRS. Both day 3 and day 7 TNFR1 levels displayed significant difference between patients with SOS versus without, but IL-6 was not different between groups (Table 8). Elevated TNFR1 was further correlated with day 3 SOS biomarkers and it was found to be associated with HA and ST2, while not with L-Ficolin (Table 18). This may imply that early increased levels of HA and ST2 are indicators of elevated inflammation as well.

Since TMA shares certain features with SOS, including platelet refractoriness and fluid retention (25), the association between day 3 biomarker levels and TMA was examined. Only two patients developed TMA, one in the SOS group and one in the no SOS group and no association between biomarker levels and TMA was found (Table 19).

SOS can cause rapid weight resulting in respiratory failure, which is also a feature of IPS. Therefore, biomarkers levels on day 3 were examined for an association with IPS. Only two patients had IPS, one in the SOS group and 1 in the no SOS group and only L-Ficolin was associated with IPS (P=0.032) (Table 20).

Given that SOS pathogenesis is a combination of endothelial damages and leukocyte infiltration due to alloreactivity, biomarker association was investigated with overall GVHD grades. Two (20%) patients developed GVHD in the SOS group and 12 (19.3%) developed GVHD in the no SOS group (Table 17). L-Ficolin was associated with GVHD while HA and ST2 were not (Table 21). Of note, neither day 3 IL-6 nor day 3 TNFR1 was associated with GVHD (Table 22).

Infections commonly occur post-HCT, however there was neither difference between patients with SOS versus without in the rate of infections grade 3 or more (Table 17) nor association for any of biomarkers on day 3 with infections (Table 23).

Associations between high and low biomarkers and SOS incidence were next calculated in univariate and multivariate Cox proportional hazards regression models (Table 4). All three biomarkers were significantly associated with SOS incidence in univariate Cox analysis, and L-Ficolin and ST2 remained significant in multivariate analysis after adjustment for the three significant clinical covariates between SOS positive and negative groups. Subcategorized age (all), transplant source, and GVHD prophylaxis were not significant in the multivariate analysis. However, in multivariate analysis, younger age patients (0 to <3 years) were at higher risk of developing SOS compared to patients aged ≥10 to <16 years (P=0.037) (Table 4). Since biomarkers values are not different in the four age categories in the recipients who did not developed SOS (Table 24), it might suggest that the correlation seen with age is due to the higher rate of SOS in younger children rather than an age effect itself. HA had an increased hazard ratio that did not reach significance in multivariate analysis suggesting it is strongly correlated to the two other biomarkers as shown in Table 9. In multivariate analyses, patients with low L-Ficolin or high ST2 had an independent increased risk of developing SOS with P-value of 0.023 and 0.008, respectively (Table 4). These data suggest that L-Ficolin and ST2 biomarkers measured as early as day 3 post-HCT are independent predictors of future development of SOS in multivariate analysis using this contemporary cohort.

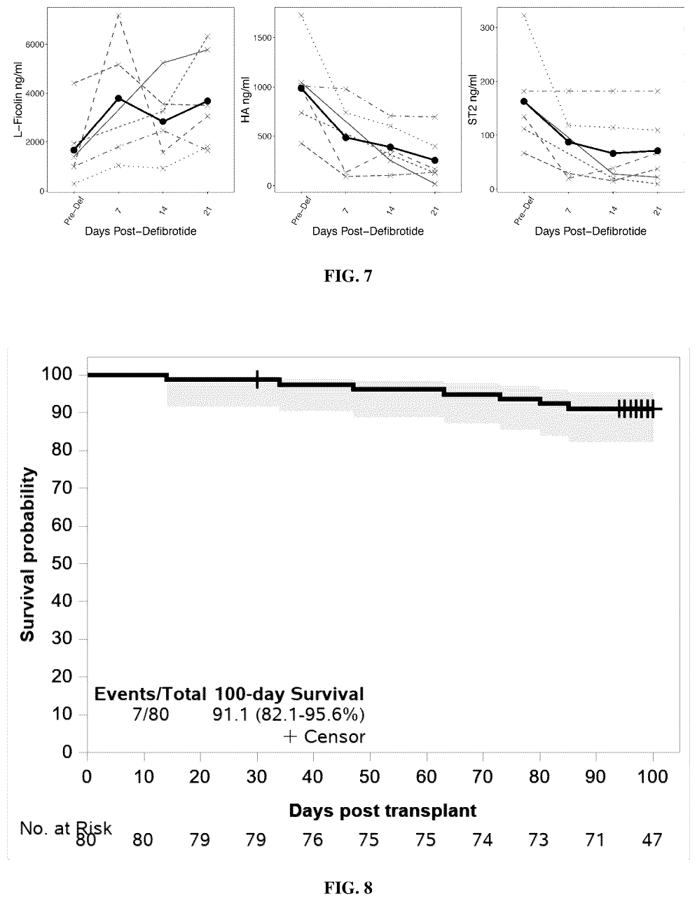

Monitoring biomarkers following defibrotide treatment. Six out of the ten patients diagnosed with SOS were treated with defibrotide, and the three biomarkers were measured at pre-defibrotide and 7-, 14-, and 21-days after defibrotide initiation. FIG. 7 shows the trend in changes from pre-defibrotide to day 21 post-defibrotide individually and on average. The inventors calculated the rate of change for biomarkers post-defibrotide treatment using general linear mixed effect models and found a significant increase in L-Ficolin values (+528 ng/ml, P=0.039) and a decrease in HA and ST2 values [HA, −233 ng/ml, P=0.019; ST2, −30 ng/ml, P=0.037] (Table 5). These data suggest that following treatment with defibrotide biomarkers values normalized toward values seen in patients without SOS.

Early identification with objective proteomic risk biomarkers may improve monitoring and management of SOS with the goal of decreasing risk of MOF. Studies were conducted for measurements of L-Ficolin, HA, and ST2 at days 3 and 7 post-HCT in a prospective cohort of 80 pediatric patients to evaluate NPV and PPV of these risk biomarkers for SOS occurrence as recommended in the 2014 NIH Consensus development project on biomarkers (20, 21). Further, these three assays have been verified for analytic validity (precision/accuracy) on one platform and have been validated in Clinical Laboratory Improvement Amendments (CLIA) settings. Although vascular cell adhesion molecule-1 (VCAM1) was a risk biomarker in the retrospective cohorts, it was not pursued as a qualifying assay due to the poor intra and inter-variabilities which assess the reproducibility of the ELISA assay itself.

This is the only prospective pediatric study conducted to assess SOS risk biomarkers. Identification of cutpoints for the three biomarkers was performed solely in one retrospective cohort (22). It was found that when the three markers were measured prospectively at day 3 post-HCT, prior to any clinical signs of SOS, they had sensitivity of 80% and specificity of 73% for estimating risk of SOS development which achieve similar predictability power than the one estimated in the retrospective cohort that was matched 1 to 1 for cases and controls (22). Importantly, Cox proportional hazards regression models showed that the markers predicted SOS independently of clinical covariates that were significantly different in patients with SOS versus without (age, transplant source, and GVHD prophylaxis). Notably, younger age (<3 years) remained significant in multivariate analysis as compared to the 10 to 16 years category, underpinning the need to closely monitor SOS in this younger population as has been suggested by the PALISI & PTCTC working group (5). The three biomarkers at day 3 also predicted SOS independently of commonly measured laboratory test of the liver function. Using the combination of the three-biomarker into a score, recipients with a positive score were 9.3 times more likely to develop SOS (95% CI 2.1-41.8, P=0.0008) (FIG. 4). This suggests that L-Ficolin, HA, and ST2 individually and dichotomized by high and low thresholds or as a combined three-biomarker score predict subclinical SOS disease several days before the diagnosis of SOS is made.

These three biomarkers may not predict other early complications. Notably, ROC analyses for the three biomarkers showed higher AUCs at day 3 compared to day 7, particularly for ST2 which might be explained by the fact that ST2 is a confounding marker of GVHD risk when measured at or after day 7 (26). However, ST2 analyzed as a continuous variable at day 7 was not correlated to GVHD which is concordant with previous studies that showed that ST2 levels pre-HCT or earlier post-HCT were correlated with nonrelapse mortality (NRM) but association with GVHD per se was either not checked or significant only when landmark analysis included ST2 measurements at days 7, 14, and 21 were performed (26-28). In this prospective cohort, IL-6 and TNFR1 were evaluated early post-HCT and although TNFR1 was associated with SOS development and correlated to HA and ST2, neither IL-6 nor TNFR1 was associated with GVHD as has been shown in a previous prospective pediatric cohort focused on analyzing NRM and GVHD (28). HA and ST2 at day 3 were not significantly associated with TMA, IPS, or infection in this prospective cohort.

Pathogenesis of the three markers although multifactorial includes endothelial injury. Indeed, L-Ficolin, a major plasma complement-activating pattern-recognition lectin, is synthesized in the liver, secreted into the bloodstream, and is involved in homeostatic clearance of mitochondria (29). In SOS patients, its concentrations are decreased, suggesting a homeostatic clearance deficiency. Interestingly, L-Ficolin was correlated with GVHD and IPS suggesting a role on alloreactivity not previously described. L-Ficolin has been shown to bind to Toll-like receptor 4 on macrophages and dendritic cells and promote their antigen-presentation to CD8⁺ T cells (30). HA is produced by mesenchymal cells, and its levels are maintained by a receptor dependent removal mechanism in the sinusoidal endothelial cells of the liver. Systemic HA levels are regarded as a direct marker of hepatic sinusoidal endothelial cell function and elevated concentrations are associated with SOS (22, 31). ELISA measures soluble ST2, acting as a decoy receptor for IL-33 (32). In a HCT preclinical model, ST2 has been shown to be initially secreted by endothelial cells and later by alloreactive T cells (33).

The markers were also interrogated as predictors of OS within 100 days post-HCT. L-Ficolin, HA, and ST2 using cutpoints as risk biomarkers were also found to be prognostic markers of OS. Although the SOS incidence was low, the severity and development to MOF in SOS patients was high. Using the three-biomarker score, recipients with a positive score were 5.5 times more likely to die by day 100 (95% CI 1.1-28.4, P=0.0219) than those with three-biomarker negative score (FIG. 6).

Six out of the ten patients diagnosed with SOS received defibrotide while the other patients were not able to receive the treatment due to contraindications, particularly bleeding. This highlights the importance of early diagnosis based on revised criteria and early intervention (15, 16, 34). Five of the SOS patients died within 100 days. In two randomized trials of defibrotide for the prevention of SOS, OS was not different between placebo and defibrotide groups (10, 11). The three markers were further tested as potential monitoring markers of response to defibrotide treatment and showed a trend towards normalization to non-SOS patient levels as early as 7 days post-treatment. If validated in a larger cohort, it could potentially justify defibrotide treatment for a shorter time course than the current 21 days.

In this contemporary prospective cohort of 80 pediatric patients with known pre-HCT risk factors for SOS, 10 patients developed SOS resulting in a 12.5% incidence which is lower than the 20% reported in the placebo group from the defibrotide prophylaxis study which might be explain by more permissive inclusion criterion as compared to the defibrotide study (10). It may also be explained by the changes to conditioning regimens and better supportive care since 2012. Since PPV is dependent on the disease incidence, it might explain its lower value. A recent analysis of data from 15 pediatric centers associated the magnitude of intravenous busulfan exposure with the development of SOS in children and young adults undergoing myeloablative allogeneic hematopoietic cell transplantation (35). A CIBMTR study found an association between SOS risk and centers performing busulfan-based myeloablative conditioning regimens guided by pharmacokinetic monitoring rather than using the target level (36). Although preparative regimen was not a significant parameter in this study, AUC was not reported in this de-identified cohort and adjustment for this parameter was not possible. In the present study, SOS diagnosis was determined using the modified Seattle criteria as they were the criteria used for defibrotide FDA approval in 2017. However, anicteric SOS forms that are frequent in children may have been underestimated (15, 16). Newer diagnostic scorings were not looked at retrospectively in this de-identified prospective cohort. Although there was no association between biomarker levels and TMA in this cohort, it is to note that TMA is a challenging clinical diagnosis and not all centers routinely screen for it. Further, in a recent study administering defibrotide for prophylaxis of TMA, high ST2 correlated with SOS diagnosis and with the only patient who died (37). Although the present study had 27% of false positives for a day 3 test, on average 15 days before the diagnosis, the possibility of overtreatment here is not a significant concern for three reasons: (i) the present study used stringent criteria for the diagnosis of SOS, and it is likely that some of these false positives are anicteric SOS which is frequent in children, and the new criteria can be used in an interventional preemptive trial; (ii) the treatment which will be defibrotide has low to no toxicity particularly when given earlier; and (iii) the cost of the drug is a lesser concern in a pediatric population.

In this prospective cohort study, three plasma biomarkers of endothelial damage measured noninvasively, three days after HCT, were associated with SOS occurrence and OS. Assessing these markers could stratify patients at high risk for SOS who may benefit from preemptive intervention with defibrotide.

### Example 2-Materials and Methods

Patients. Pediatric patients (up to 22 years) of any sex, race, and ethnicity undergoing HCT for any indication who fulfill clinical criteria for high-risk of SOS at enrollment (i.e., history of hepatic disease, conditioning with busulfan or total body irradiation, ≥2 HCT) were eligible for enrollment. Patients were recruited across 4 centers from 2017 to 2021. SOS was diagnosed based on the modified Seattle criteria (12). Table 6 shows the SOS severity scale used in the trial (38).

Sample collection, processing, and ELISA. Plasma samples were prospectively collected at days 3 and 7 post-HCT, prior to the onset of SOS. A window of +/−3 days was authorized to avoid shipment on weekends; however, day 0 samples were taken several hours after the graft transfusion. ELISA procedures and parameters are described in supplemental methods and Table 7.

TABLE 1
Patients' Demographics

| Characteristic | Overall (N = 80) | | SOS N (N = 70) | | SOS Y (N = 10) | | P-value |
|---|---|---|---|---|---|---|---|
| Age category n (%) | | | | | | | 0.016^{†} |
| 0 to <3 | 17 | (21.25%) | 11 | (15.71%) | 6 | (60.00%) | |
| ≥3 to <10 | 21 | (26.25%) | 20 | (28.57%) | 1 | (10.00%) | |
| ≥10 to <16 | 26 | (32.50%) | 25 | (35.71%) | 1 | (10.00%) | |
| ≥16 to ≤ 22 | 16 | (20.00%) | 14 | (20.00%) | 2 | (20.00%) | |
| Underlying Disease n (%) | | | | | | | 0.21 |
| ALL | 20 | (25.00%) | 18 | (25.71%) | 2 | (20.00%) | |
| AML | 19 | (23.75%) | 17 | (24.29%) | 2 | (20.00%) | |
| MDS/MPS | 2 | (2.50%) | 1 | (1.43%) | 1 | (10.00%) | |
| Other Malignancies | 19 | (23.75%) | 18 | (25.71%) | 1 | (10.00%) | |
| Immune Deficiencies | 8 | (10.00%) | 5 | (7.14%) | 3 | (30.00%) | |
| Inherited Disorders | 5 | (6.25%) | 4 | (5.71%) | 1 | (10.00%) | |
| SAA | 5 | (6.25%) | 5 | (7.14%) | 0 | (0%) | |
| Other non-malignant disease | 2 | (2.50%) | 2 | (2.86%) | 0 | (0%) | |
| Donor type n (%) | | | | | | | 0.62 |
| Allo-related | 33 | (41.2%) | 29 | (41.4%) | 4 | (40%) | |
| Allo-unrelated | 39 | (48.8%) | 33 | (47.1%) | 6 | (60%) | |
| Autologous | 8 | (10%) | 8 | (11.5%) | 0 | (0%) | |
| Donor HLA match n (%) | | | | | | | 0.64 |
| Yes | 61 | (84.7%) | 53 | (85.5%) | 8 | (80%) | |
| Transplant Source n (%) | | | | | | | 0.038^{†} |
| PBSC | 12 | (15%) | 11 | (15.7%) | 1 | (10%) | |
| Marrow | 58 | (72.5%) | 53 | (75.7%) | 5 | (50%) | |
| Cord | 10 | (12.5%) | 6 | (8.6%) | 4 | (40%) | |
| Conditioning Regimen n (%) | | | | | | | 0.45 |
| Auto^{$} | 8 | (10.00%) | 8 | (11.43%) | 0 | (0%) | |
| BU/CY | 26 | (32.50%) | 24 | (34.29%) | 2 | (20.00%) | |
| BU/FLU | 19 | (23.75%) | 14 | (20.00%) | 5 | (50.00%) | |
| CY/FLU | 2 | (2.50%) | 2 | (2.86%) | 0 | (0%) | |
| CY/TBI | 13 | (16.25%) | 11 | (15.71%) | 2 | (20.00%) | |
| FLU/MEL | 6 | (7.50%) | 5 | (7.14%) | 1 | (10.00%) | |
| Other allo^{#} | 6 | (7.50%) | 6 | (8.57%) | 0 | (0%) | |
| GVHD prophylaxis n (%) | | | | | | | 0.002^{†} |
| Tacro or CsA/MTX | 25 | (34.72%) | 24 | (38.71%) | 1 | (10.00%) | |
| ATG* | 15 | (20.83%) | 9 | (14.52%) | 6 | (60.00%) | |
| Alemtuzumab | 22 | (30.56%) | 21 | (33.87%) | 1 | (10.00%) | |
| PTCY | 6 | (8.33%) | 6 | (9.68%) | 0 | (0%) | |
| Others^{§} | 4 | (5.56%) | 2 | (3.23%) | 2 | (20.00%) | |

Footnotes:
ALL, Acute Lymphoblastic Leukemia;
AML, Acute myeloid leukemia;
MDS/MPS, myelodysplastic/myeloproliferative syndrome;
SAA, Severe aplastic anemia;
HLA, Human leukocyte Antigen;
PBSC, Peripheral Blood Stem Cells;
BU, Busulfan (AUC > 900);
CY, Cyclophosphamide;
FLU, Fludarabine;
TBI, Total Body Irradiation >= 1200 cGy;
MEL, Melphalan;
GVHD, Graft-Versus-Host Disease;
Tacro, Tacrolimus;
CsA, Cyclosporine;
MTX, Methotrexate;
MMF, Mycophenolate Mofetil;
ATG, Anti-thymocyte globulin
^{$}Auto-HCT conditioning regimens included melphalan, etoposide, carboplatin, Thiotepa + Carboplatin, Busulfan + Melphalan.
^{#}Other allo conditioning: TBI/Etoposide, FLU/TBI, CY/Aracytine.
*Including one patient with CD34 selection
^{§}with MMF n = 3 (SOS = Y n = 1), with bortezomib n = 1 (SOS = Y n = 1).
^{†}P-value < 0.05, P-value comparisons across SOS categories are based on fisher's exact test for categorical variables; p-values for continuous variables are based on the Wilcoxon Rank Sum test for median.

TABLE 2
Outcomes' Characteristics.

| | Overall | SOS N | SOS Y | | |
|---|---|---|---|---|---|
| Outcomes | (N = 80) | (N = 70) | (N = 10) | | P-value |
| Days after transplant to | N/A | N/A | 19 | (9, 34) | N/A |
| SOS onset, Median (range) | | | | | |
| SOS grades n (%) | | | | | N/A |
| Mild | | N/A | 0 | (0%) | |
| Moderate | | N/A | 0 | (0%) | |
| Severe | | N/A | 10 | (100%) | |
| Deaths by day 100 n (%) | | | | | <0.001^{†} |
| Yes | 7 (8.8%) | 2^{⋄} (2.9%) | 5 [symbol] | (50%) | |

Footnotes:
^{⋄}Causes of death in the no SOS group were respectively, 1 IPS/fungal pneumonia and 1 disease progression. [symbol]
Causes of death in the yes SOS group were respectively, 3 SOS, 1 MOF, and 1 cardiac arrest.
^{†}P-value <0.05, P-value comparisons across SOS categories are based on fisher's exact test for categorical variables; p-values for continuous variables are based on the Wilcoxon Rank Sum test for median.

TABLE 3
Parameter estimates and p-values in the Cox proportional hazards
regression model with three binary biomarkers to create a
three-biomarker score.

| | | 95% | | | |
|---|---|---|---|---|---|
| | | confidence | | | |
| | Beta | interval (CI) | | | |
| Parameter | estimate | for beta | | P-value | Note of score |
| L-Ficolin | 2.47 | 0.85 | 4.10 | 0.003^{†} | Low/High - 1/0 |
| at Day +3 | | | | | |
| HA at Day +3 | 1.12 | −0.24 | 2.48 | 0.11 | High/Low - 1/0 |
| ST2 at Day +3 | 2.11 | 0.56 | 3.67 | 0.008^{†} | High/Low - 1/0 |

Note^{†}:
p-value < 0.05.

TABLE 4
Univariate (top) and multivariate (bottom) Cox regression analyses
for SOS incidence.

| | | | |
|---|---|---|---|
| | Hazard | | |
| | ratio | | |
| Variable | (HR) | 95% CI of HR | P-value |

Univariate

| | | | | |
|---|---|---|---|---|
| L-Ficolin Low vs High | 9.1 | 2.5 | 32.5 | 0.0007^{†} |
| HA High vs Low | 6.4 | 1.8 | 22.9 | 0.0039^{†} |
| ST2 High vs Low | 5.0 | 1.4 | 17.9 | 0.0124^{†} |

Multivariate

| | | | | |
|---|---|---|---|---|
| Age | | | | 0.09 |
| ≥3 to <10 years vs | 0.97 | 0.03 | 30.4 | 0.99 |
| 0 to <3 years | | | | |
| ≥10 to <16 years vs | 0.009 | 0.000 | 0.7 | 0.037^{†} |
| 0 to <3 years | | | | |
| ≥16 to ≤ 22 years vs | 2.8 | 0.09 | 85.0 | 0.56 |
| 0 to <3years | | | | |
| Transplant Source | | | | 0.995 |
| Marrow vs PBSC | 1.5 | 0.00 | 2.4E6 | 0.95 |
| Cord vs PBSC | 1.4 | 0.00 | 2.5E6 | 0.96 |
| GVHD prophylaxis | | | | 0.10 |
| ATG vs Tacro or CsA/MTX | 12.3 | 0.8 | 184.5 | 0.07 |
| Alemtuzumab vs Tacro or | 0.06 | 0.001 | 3.9 | 0.19 |
| CsA/MTX | | | | |
| Other vs Tacro or CsA/MTX | 0.3 | 0.006 | 13.4 | 0.51 |
| PTCY vs Tacro or CsA/MTX | 0.000 | 0.000 | N/A | 0.994 |
| L-Ficolin Low vs High | 21.3 | 1.5 | 295.0 | 0.0225^{†} |
| HA High vs Low | 10.4 | 0.8 | 134.7 | 0.0741 |
| ST2 High vs Low | 284.9 | 4.3 | 1.9E4 | 0.0084^{†} |

Note^{†}:
p-value < 0.05

TABLE 5
Rate of change for biomarkers post-defibrotide treatment.

| | Estimate | Estimate | 95% CI | | |
|---|---|---|---|---|---|
| | (raw value) | (log transformed) | (log transformed) | | P-value |
| L-Ficolin | +528 | +0.28 | +0.02 | +0.54 | 0.039* |
| HA | −233 | −0.53 | −0.93 | −0.13 | 0.019* |
| ST2 | −30 | −0.37 | −0.70 | −0.03 | 0.037* |

Note:
p-value were estimated on natural logarithm transformed L-Ficolin, HA, or ST2 values
Note *:
p-value < 0.05

Statistical Analyses. Differences in patient group characteristics were assessed using cither Fisher's exact tests or Wilcoxon Rank Sum tests. Differences for biomarkers and liver functions between SOS yes or no, were checked by two-sample t-test or Wilcoxon Rank Sum test. Correlations between biomarkers and liver functions or inflammatory cytokines were measured using Pearson correlation coefficients. Receiver operating characteristic (ROC) curves were generated on days 3 and 7. The optimal biomarkers cutpoints were obtained from biomarkers values of all three cohorts from our previous retrospective study (22) based on exhaustive grid search and Youden's index selection. First, each biomarker was set to be in a clinically relevant range of values (L-Ficolin: 500 to 1200; HA: 50 to 220; ST2:30 to 50, respectively). Youden's index was then calculated for each combination of L-Ficolin, HA, and ST2. The cutpoints were determined by searching the combination that could reach the higher Youden's index. Kaplan-Meier method based cumulative incidence curves for SOS by day 35 and overall survival (OS) curves by day 100 were produced with day 3 individual biomarker or combined three biomarkers using the aforementioned cutpoints to determine high- and low-risk groups. The Cox proportional hazards regression score was used to generate two combined biomarkers groups for the cumulative incidence and OS curves. First, three binary day 3 biomarkers (L-Ficolin low/high, HA high/low, ST2 high/low were coded as I/O) were incorporated into Cox proportional hazards regression to get the beta estimates for each biomarker. Second, score was defined as beta*X for each patient. Third, two combined biomarkers groups: three-biomarker positive score and three-biomarker negative score were formed by score>0 and score=0, respectively. Comparison between groups was evaluated using Gray K-sample tests or log-rank tests. Associations between biomarkers and inflammatory cytokines, thrombotic microangiopathy (TMA), idiopathic pneumonia syndrome (IPS), graft-versus-host disease (GVHD), and infections were examined by logistic regression models. Univariate and multivariate Cox regression analyses evaluated the effect of day 3 biomarkers adjusted for significant clinical characteristics. For biomarker monitoring post-defibrotide, the rate of change was estimated by fitting general linear mixed random intercepts and slopes effect models (using PROC MIXED) with an unstructured correlation for time. Statistical significance was defined as a P value less than 0.05.

Study design and oversight. The study design and samples collection are summarized in FIG. 1. The study was approved by the Institutional Review boards of all institutional participating centers. Informed consent was obtained from all patients or their legal guardians.

Study Patients. Detailed eligibility criteria for the study are described below.

Inclusion Criteria. Age≤25 years undergoing HCT for any reason who fulfill any ONE of the following criteria:
1. History of hepatic disease as defined by:
   a. Viral hepatitis (i.e., hepatitis C virus [HCV])
   b. Liver tumor before HCT
   c. Hepatic fibrosis or cirrhosis before HCT as proven by liver biopsy
   d. High aspartate aminotransferase (AST) (>2× ULN) before HCT (pre-transplant evaluation)
   e. High alanine transaminase (ALT) (>2× ULN) before HCT
   f. High bilirubin (>1.2× ULN) before HCT
2. HCT high-risk features including:
   a. Conditioning with high-risk modalities including:
      i. Busulfan (BU)-containing regimen particularly with oral BU +cyclophosphamide
      ii. TBI-containing regimen, particularly cyclophosphamide+total-body irradiation (TBI)
   b. ≥2 HCT
   c. Allo-HCT for leukemia > or =second relapse
   d. Unrelated donor (URD) HCT
   e. Human leukocyte antigen (HLA) mismatch HCT (less than 10 of 10 for bone marrow/peripheral blood stem cell [BM/PBSC] or anything less than 6 of 6 for UCB)
   f. Use of sirolimus+tacrolimus prophylaxis for GVHD
3. High-risk disease states including:
   a. Juvenile myelo-monocytic chronic leukemia (JMML)
   b. Primary hemophagocytic lymphohistiocytosis (HLH)
   c. Adrenoleukodystrophy
   d. Osteopetrosis
4. Other high-risk features including:
   a. Prior treatment with gemtuzumab ozogamicin
   b. Use of hepatotoxic drugs 1 month before HCT and during HCT
   c. Iron overload (i.e., thalassemia/sickle cell) with serum ferritin >1000 ng/ml
   d. Deficit of ATIII, T-PA (i.e., <30% normal values), and resistance to activated protein C if clinical indication (these values do not have to be specifically checked if no clinical history)
   e. Young age <2 years but more than 1 month

Exclusion Criteria. Patients who are transplanted but do not fulfill any of the above-mentioned criteria.

ELISA. Antibody pairs were purchased as described in Table 7. Proteins were measured in samples using commercially available enzyme-linked immunosorbent assays (ELISA) and following the manufacturer' recommendations and using a sequential ELISA approach previous described. All samples and standards were tested in duplicate. All washes were performed using the Aquamax 2000 plate washer (Molecular Devices, Sunnyvale, CA). Absorbance was measured immediately after termination of the substrate reaction using a SpectraMax ABS Plus plate reader and results were calculated using SoftMax Pro Version 7.1 (Molecular Devices, Sunnyvale, CA).

TABLE 6
SOS Severity Scale.

| | SOS Grade | | |
|---|---|---|---|
| Criteria | Mild | Moderate | Severe |
| Bilirubin mg/dL | 2.0-3.0 | 3.1-5.0 | >5.0 |
| Liver function | <3X normal | 3-5 normal | >5X normal |
| Weight above baseline | 2% | 2.1-5% | >5% |
| Renal function | normal | <2X normal | ≥2X normal |
| Rate of change, days | Slow (>6) | Moderate (4-5) | Rapid (<3) |

TABLE 7
Details of ELISAs used for protein testing.

| | | Commercial | | | | | |
|---|---|---|---|---|---|---|---|
| Protein | | ELISA | Plasma | | | | |
| name | Description | provider | dilution | LLOD | | ULOD | |
| L- | Ficolin-2 | Hycult | 1:20 | 16 | ng/ml | 20000 | ng/ml |
| Ficolin | | Biotech | | | | | |
| HA | Hyaluronic | Corgenix | 1:1 | 30 | ng/ml | 4000 | ng/ml |
| | Acid | | | | | | |
| ST2 | Stimulation- | R&D | 1:50 | 1 | ng/ml | 200 | ng/ml |
| | 2, IL-33 | Quantikine | | | | | |
| | receptor | | | | | | |
| IL-6 | Interleukin-6 | R&D Duoset | 1:1 | 0.5 | pg/ml | 2000 | pg/ml |
| TNFR1 | Tumor | R&D Duoset | 1:25 | 100 | pg/ml | 20000 | pg/ml |
| | Necrosis | | | | | | |
| | Factor | | | | | | |
| | Receptor-1 | | | | | | |

TABLE 8
Biomarkers Descriptive Statistics.

| | SOS Yes (n = 10) | | | SOS No (n = 70) | | | |
|---|---|---|---|---|---|---|---|
| | | Mean ± | | | Mean ± | | |
| | | Standard | Median | | Standard | Median | |
| Variable | N | Deviation | (Min-Max) | N | Deviation | (Min-Max) | P-value |
| Biomarkers | | | | | | | |
| L-Ficolin Day +3 | 10 | 2399 ± 2036 | 2002 | 70 | 3722 ± 2162 | 3321 | 0.07* |
| | | | (158-5932) | | | (848-14373) | |
| HA Day +3 | | 465 ± 572 | 216 | | 167 ± 236 | 78 | 0.004^{#†} |
| | | | (67-1870) | | | (17-1422) | |
| ST2 Day +3 | | 63 ± 65 | 36 | | 26 ± 18 | 20 | 0.036^{#†} |
| | | | (6-202) | | | (3-99) | |
| L-Ficolin Day +7 | 10 | 2592 ± 2553 | 1721 | 69 | 3596 ± 2048 | 3197 | 0.09^{#} |
| | | | (289-7176) | | | (357-12038) | |
| HA Day +7 | | 751 ± 745 | 351 | | 195 ± 218 | 117 | 0.005^{#†} |
| | | | (82-2043) | | | (27-1176) | |
| ST2 Day +7 | | 72 ± 101 | 26 | | 30 ± 24 | 21 | 0.47^{#} |
| | | | (8-322) | | | (6-113) | |
| IL6 Day +3 | 10 | 215 ± 509 | 8 | 70 | 37 ± 64 | 18 | 0.72^{#} |
| | | | (0-1602) | | | (0-345) | |
| TNFR1 Day +3 | | 3913 ± 5609 | 1809 | | 1044 ± 659 | 906 | <.001^{#†} |
| | | | (824-19575) | | | (184-5242) | |
| IL6 Day +7 | 10 | 244 ± 657 | 16 | 69 | 53 ± 78 | 26 | 0.82^{#} |
| | | | (0-2107) | | | (0-421) | |
| TNFR1 Day +7 | | 6948 ± 13725 | 2330 | | 1218 ± 576 | 1086 | 0.0002^{#†} |
| | | | (1129-45816) | | | (184-3116) | |
| Liver Functions | | | | | | | |
| Bilirubin Day +3 | 10 | 0.63 ± 0.39 | 0.6 | 70 | 0.47 ± 0.35 | 0.4 | 0.22^{#} |
| | | | (0.2-1.3) | | | (0.0-1.5) | |
| AST Day +3 | | 31 ± 14 | 31 | | 37 ± 27 | 28 | 0.80^{#} |
| | | | (12-52) | | | (12-154) | |
| ALT Day +3 | | 33 ± 28 | 22 | | 40 ± 36 | 26 | 0.71^{#} |
| | | | (10-102) | | | (5-176) | |
| Bilirubin Day +7 | 10 | 0.80 ± 0.66 | 0.55 | 69 | 0.45 ± 0.26 | 0.4 | 0.17^{#} |
| | | | (0.2-2) | | | (0.0-1.4) | |
| AST Day +7 | | 21 ± 7 | 22 | | 35 ± 34 | 29 | 0.08^{#} |
| | | | (12-34) | | | (9-253) | |
| ALT Day +7 | | 21 ± 9 | 18 | | 45 ± 55 | 23 | 0.24^{#} |
| | | | (8-37) | | | (5-276) | |

Note*:
Using the two-sample t-test
Note^{#}:
Using the Wilcoxon Rank Sum test
Note^{†}:
P-value < 0.05

TABLE 9
Pearson Correlation between Biomarkers.
Pearson Correlation Coefficients
Prob > |r| under H0: Rho = 0
Number of Observations

| | L-Ficolin | | | L-Ficolin | | |
|---|---|---|---|---|---|---|
| | Day +3 | HA Day +3 | ST2 Day +3 | Day +7 | HA Day +7 | ST2 Day +7 |
| L-Ficolin | 1.00000 | −0.33312 | −0.15587 | 0.72525 | −0.36196 | −0.11837 |
| Day +3 | | 0.0025* | 0.1674 | <.0001* | 0.0010* | 0.2988 |
| | 80 | 80 | 80 | 79 | 79 | 79 |
| HA Day +3 | | 1.00000 | 0.69013 | −0.33571 | 0.64033 | 0.70611 |
| | | | <.0001* | 0.0025* | <.0001* | <.0001* |
| | | 80 | 80 | 79 | 79 | 79 |
| ST2 Day +3 | | | 1.00000 | −0.19167 | 0.48597 | 0.74086 |
| | | | | 0.0906 | <.0001* | <.0001* |
| | | | 80 | 79 | 79 | 79 |
| L-Ficolin | | | | 1.00000 | −0.42077 | −0.19647 |
| Day +7 | | | | | 0.0001* | 0.0827 |
| | | | | 79 | 79 | 79 |
| HA Day +7 | | | | | 1.00000 | 0.55123 |
| | | | | | | <.0001* |
| | | | | | 79 | 79 |
| ST2 Day +7 | | | | | | 1.00000 |
| | | | | | | 79 |

Note*:
p-value < 0.05.

TABLE 10
Sensitivity, specificity, PPV, and NPV using optimal cutpoints in
retrospective cohort.

| | Estimate | Standard | 95% Confidence | |
|---|---|---|---|---|
| Statistic | (%) | Error (%) | Limits (%) | |
| Sensitivity | 64.71 | 8.20 | 48.64 | 80.77 |
| Specificity | 74.19 | 7.86 | 58.79 | 89.60 |
| Positive Predictive Value | 73.33 | 8.07 | 57.51 | 89.16 |
| Negative Predictive Value | 65.71 | 8.02 | 49.99 | 81.44 |

TABLE 11
Sensitivity, specificity, PPV, and NPV of individual biomarkers
using cutpoints in prospective cohort.

| Bio- | | Estimate | Standard | 95% Confidence | |
|---|---|---|---|---|---|
| marker | Statistic | (%) | Error (%) | Limits (%) | |
| L-Ficolin | Sensitivity | 40.00 | 15.49 | 9.64 | 70.36 |
| | Specificity | 94.29 | 2.77 | 88.85 | 99.72 |
| | Positive | 50.00 | 17.68 | 15.35 | 84.65 |
| | Predictive | | | | |
| | Value | | | | |
| | Negative | 91.67 | 3.26 | 85.28 | 98.05 |
| | Predictive | | | | |
| | Value | | | | |
| HA | Sensitivity | 60.00 | 15.49 | 29.64 | 90.36 |
| | Specificity | 82.86 | 4.50 | 74.03 | 91.69 |
| | Positive | 33.33 | 11.11 | 11.56 | 55.11 |
| | Predictive | | | | |
| | Value | | | | |
| | Negative | 93.55 | 3.12 | 87.43 | 99.66 |
| | Predictive | | | | |
| | Value | | | | |
| ST2 | Sensitivity | 40.00 | 15.49 | 9.64 | 70.36 |
| | Specificity | 90.00 | 3.59 | 82.97 | 97.03 |
| | Positive | 36.36 | 14.50 | 7.94 | 64.79 |
| | Predictive | | | | |
| | Value | | | | |
| | Negative | 91.30 | 3.39 | 84.66 | 97.95 |
| | Predictive | | | | |
| | Value | | | | |

TABLE 12
Correlation between biomarker levels and maximum total serum bilirubin

| Biomarker and Time Point | n | Pearson's r | p-value |
|---|---|---|---|
| L-Ficolin at Day +3 | 80 | −0.1250 | 0.27 |
| HA at Day +3 | 80 | 0.2468 | 0.027* |
| ST2 at Day +3 | 80 | 0.4298 | <.001* |

Note
*p-value < 0.05.

TABLE 13
Correlation between biomarker levels and total bilirubin at day 3.

| Biomarker and Time Point | n | Pearson's r | p-value |
|---|---|---|---|
| L-Ficolin at Day +3 | 80 | 0.0398 | 0.73 |
| HA at Day +3 | 80 | 0.0430 | 0.71 |
| ST2 at Day +3 | 80 | 0.3066 | 0.006* |

Note
*p-value < 0.05.

TABLE 14
Correlation between biomarker levels and alkaline phosphatase at day 3.

| Biomarker and Time Point | n | Pearson's r | p-value |
|---|---|---|---|
| L-Ficolin at Day +3 | 79 | 0.0040 | 0.97 |
| HA at Day +3 | 79 | −0.0079 | 0.95 |
| ST2 at Day +3 | 79 | −0.2260 | 0.045* |

Note
*p-value < 0.05.

TABLE 15
Correlation between biomarker levels and AST at day 3.

| Biomarker and Time Point | n | Pearson's r | p-value |
|---|---|---|---|
| L-Ficolin at Day +3 | 80 | −0.1366 | 0.23 |
| HA at Day +3 | 80 | 0.2010 | 0.07 |
| ST2 at Day +3 | 80 | −0.0433 | 0.70 |

TABLE 16
Correlation between biomarker levels and ALT at day 3.

| Biomarker and Time Point | n | Pearson's r | p-value |
|---|---|---|---|
| L-Ficolin at Day +3 | 80 | −0.0790 | 0.49 |
| HA at Day +3 | 80 | 0.1452 | 0.20 |
| ST2 at Day +3 | 80 | −0.0496 | 0.66 |

TABLE 17
Demographics for other post-HCT complications.

| | Overall | SOS N | SOS Y | |
|---|---|---|---|---|
| Characteristic | N = 80 | N = 70 | N = 10 | P-value |
| # of TMA cases n (%) | | | | 0.24 |
| No | 78 (97.5%) | 69 (98.6%) | 9 (90%) | |
| Yes | 2 (2.5%) | 1 (1.4%) | 1 (10%) | |
| # of IPS cases n (%) | | | | 0.26 |
| No | 70 (97.2%) | 61 (98.4%) | 9 (90%) | |
| Yes | 2 (2.8%) | 1 (1.6%) | 1 (10%) | |
| Acute GVHD n (%) | | | | 1.00 |
| No | 58 (80.6%) | 50 (80.7%) | 8 (80%) | |
| Yes | 14 (19.4%) | 12 (19.3%) | 2 (20%) | |
| Days after transplant to GVHD | 48 (18, 305) | 40 (18, 305) | 64 (63, 66) | 0.08 |
| onset | | | | |
| Median (range) | | | | |
| GVHD grades n (%) | | | | 0.34 |
| 0 | 58 (80.6%) | 50 (80.7%) | 8 (80%) | |
| I-Stage 1-2 Rash and no liver | 5 (6.9%) | 5 (8.1%) | 0 (0%) | |
| or gut involvement | | | | |
| II-Stage 3 rash, or stage 1 liver | 5 (6.9%) | 3 (4.8%) | 2 (20%) | |
| involvement or stage 1 gut | | | | |
| involvement | | | | |
| III-None to stage 3 skin rash with | 4 (5.6%) | 4 (6.5%) | 0 (0%) | |
| stage 2-3 liver, or stage 2-4 gut | | | | |
| involvement | | | | |
| Infection grade >= 3 n (%) | | | | 0.12 |
| Bacterial | 10 (55.56%) | 8 (53.33%) | 2 (66.67%) | |
| Bacterial and Viral | 1 (5.56%) | 0 (0%)0 | 1 (33.33%) | |
| | | (0%) | | |
| Viral | 7 (38.89%) | 7 (46.67%) | 0 (0%) | |

TMA, Thrombotic Microangiopathy; IPS, Idiopathic Pneumonia Syndrome; GVHD, Graft-Versus-Host Disease
Note
†P-value < 0.05, P-value comparisons across SOS categories are based on fisher's exact test for categorical variables; p-values for continuous variables are based on the Wilcoxon Rank Sum test for median.

TABLE 18
Correlation between biomarker levels and TNER1 at day 3.

| Biomarker and Time Point | n | Pearson's r | p-value |
|---|---|---|---|
| L-Ficolin at Day +3 | 80 | −0.1672 | 0.14 |
| HA at Day +3 | 80 | 0.5029 | <.001* |
| ST2 at Day +3 | 80 | 0.7822 | <.001* |

Note
*p-value < 0.05.

TABLE 19
Association between biomarker levels and thrombotic microangiopathy (TMA).

| Biomarker and Time | | | | | |
|---|---|---|---|---|---|
| Point | n | Beta estimate | 95% CI | | p-value |
| L-Ficolin at Day +3 | 80 | −0.00002 | −0.0007 | 0.0006 | 0.95 |
| HA at Day +3 | 80 | 0.0008 | −0.002 | 0.004 | 0.59 |
| ST2 at Day +3 | 80 | 0.010 | −0.018 | 0.038 | 0.48 |

TABLE 20
Association between biomarker levels and idiopathic pneumonia syndrome (IPS).

| Biomarker and Time | | | | | |
|---|---|---|---|---|---|
| Point | n | Beta estimate | 95% CI | | p-value |
| L-Ficolin at Day +3 | 72 | 0.00056 | 0.00005 | 0.0010 | 0.032* |
| HA at Day +3 | 72 | −0.0002 | −0.006 | 0.0053 | 0.94 |
| ST2 at Day +3 | 72 | 0.0014 | −0.042 | 0.045 | 0.95 |

Note
*p-value < 0.05.

TABLE 21
Association between biomarker levels and overall graft-versus-
host disease (GVHD) severity.

| Biomarker | | | | | |
|---|---|---|---|---|---|
| and | | Beta | | | |
| Time Point | n | estimate | 95% CI | | p-value |
| L-Ficolin at | 72 | −0.0003 | −0.0006 | −0.00008 | 0.011* |
| Day +3 | | | | | |
| HA at | 72 | −0.0001 | −0.002 | 0.0019 | 0.92 |
| Day +3 | | | | | |
| ST2 at | 72 | 0.0012 | −0.019 | 0.021 | 0.90 |
| Day +3 | | | | | |

Note
*p-value < 0.05.

TABLE 22
Association between IL-6 and TNFR1 levels and overall graft-versus-
host disease (GVHD) severity.

| Biomarker and Time | | | | | |
|---|---|---|---|---|---|
| Point | n | Beta estimate | 95% CI | | p-value |
| IL-6 at Day +3 | 72 | 0.0006 | −0.0032 | 0.0044 | 0.77 |
| TNFR1 at Day +3 | 72 | 0.00009 | −0.0004 | 0.0005 | 0.70 |

TABLE 23
Association between biomarker levels and infections.

| Biomarker and | | | | | | p- |
|---|---|---|---|---|---|---|
| Time Point | n | Beta estimate | | 95% CI | | value |
| L-Ficolin at Day +3 | 18 | | | | | 0.44 |
| | | Bacterial and Viral vs. | −0.0004 | −0.0020 | 0.0013 | 0.65 |
| | | Bacterial | | | | |
| | | Virial vs. Bacterial | 0.0003 | −0.0002 | 0.0009 | 0.25 |
| HA at Day +3 | 18 | | | | | 0.50 |
| | | Bacterial and Viral vs. | 0.0012 | −0.0028 | 0.0051 | 0.57 |
| | | Bacterial | | | | |
| | | Virial vs. Bacterial | −0.0023 | −0.0070 | 0.0024 | 0.33 |
| ST2 at Day +3 | 18 | | | | | 0.22 |
| | | Bacterial and Viral vs. | 0.0059 | −0.0268 | 0.0385 | 0.73 |
| | | Bacterial | | | | |
| | | Virial vs. Bacterial | −0.0955 | −0.2066 | 0.0156 | 0.09 |

TABLE 24
Descriptive statistics of biomarkers in subcategorized age in patients without SOS.

| | Age in patients without SOS, mean ± Standard Deviation | | | | | |
|---|---|---|---|---|---|---|
| | Overall | 0 to <3 | >=3 to <10 | >=10 to <16 | >=16 to <=22 | P- |
| Biomarker | N = 70 | N = 11 | N = 20 | N = 25 | N = 14 | value |
| L-Ficolin Day +3 | 3722 ± 2162 | 3039 ± 1909 | 3525 ± 1846 | 3864 ± 1648 | 4285 ± 3344 | 0.52 |
| HA Day +3 | 167 ± 236 | 203 ± 193 | 257 ± 354 | 124 ± 175 | 88 ± 51 | 0.13 |
| ST2 Day +3 | 26 ± 18 | 21 ± 20 | 24 ± 19 | 26 ± 19 | 32 ± 12 | 0.47 |

Note:
P-value is based on ANOVA.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Carreras E, and Diaz-Ricart M. The role of the endothelium in the short-term complications of hematopoietic SCT. Bone Marrow Transplant. 2011; 46 (12): 1495-502.
2. Carreras E. How I manage sinusoidal obstruction syndrome after haematopoietic cell transplantation. Br J Haematol. 2015; 168 (4): 481-91.
3. Mohty M, Malard F, Abecassis M, Aerts E, Alaskar A S, Aljurf M, et al. Sinusoidal obstruction syndrome/veno-occlusive disease: current situation and perspectives-a position statement from the European Society for Blood and Marrow Transplantation (EBMT). Bone Marrow Transplant. 2015.
4. Bazarbachi A H, Al Hamed R, Labopin M, Halaburda K, Labussiere H, Bernasconi P, et al. Underdiagnosed veno-occlusive disease/sinusoidal obstruction syndrome (VOD/SOS) as a major cause of multi-organ failure in acute leukemia transplant patients: an analysis from the EBMT Acute Leukemia Working Party. Bone Marrow Transplant. 2021; 56 (4): 917-27.
5. Bajwa RPS, Mahadeo K M, Taragin B H, Dvorak C C, McArthur J, Jeyapalan A, et al. Consensus Report by Pediatric Acute Lung Injury and Sepsis Investigators and Pediatric Blood and Marrow Transplantation Consortium Joint Working Committees: Supportive Care Guidelines for Management of Veno-Occlusive Disease in Children and Adolescents, Part 1: Focus on Investigations, Prophylaxis, and Specific Treatment. Biol Blood Marrow Transplant. 2017; 23 (11): 1817-25.
6. Kornblum N, Ayyanar K, Benimetskaya L, Richardson P, Iacobelli M, and Stein CA. Defibrotide, a polydisperse mixture of single-stranded phosphodiester oligonucleotides with lifesaving activity in severe hepatic veno-occlusive disease: clinical outcomes and potential mechanisms of action. Oligonucleotides. 2006; 16 (1): 105-14.
7. Richardson P G, Riches M L, Kernan N A, Brochstein J A, Mineishi S, Termuhlen A M, et al. Phase 3 trial of defibrotide for the treatment of severe veno-occlusive disease and multi-organ failure. Blood. 2016.
8. Kernan N A, Grupp S, Smith A R, Arai S, Triplett B, Antin J H, et al. Final results from a defibrotide treatment-IND study for patients with hepatic veno-occlusive disease/sinusoidal obstruction syndrome. Br J Haematol. 2018; 181 (6): 816-27.
9. Richardson P G, Corbacioglu S, Ho V T, Kernan N A, Lehmann L, Maguire C, et al. Drug safety evaluation of defibrotide. Expert Opin Drug Saf. 2013; 12 (1): 123-36.
10. Corbacioglu S, Cesaro S, Faraci M, Valteau-Couanet D, Gruhn B, Rovelli A, et al. Defibrotide for prophylaxis of hepatic veno-occlusive disease in paediatric haemopoietic stem-cell transplantation: an open-label, phase 3, randomised controlled trial. Lancet. 2012; 379 (9823): 1301-9.
11. Grupp S A C S, Kang H J, Teshima T, Zanette M, Lopez P, Vian Amber V, Pagliuca A, Richardson PG. A Phase 3, Randomized, Adaptive Study of Defibrotide (DF) Vs Best Supportive Care (BSC) for the Prevention of Hepatic Veno-Occlusive Disease/Sinusoidal Obstruction Syndrome (VOD/SOS) in Patients (pts) Undergoing Hematopoietic Cell Transplantation (HCT): Preliminary Results. Blood. 2021.
12. McDonald G B, Sharma P, Matthews D E, Shulman H M, and Thomas E D. Venocclusive disease of the liver after bone marrow transplantation: diagnosis, incidence, and predisposing factors. Hepatology. 1984; 4 (1): 116-22.
13. Jones R J, Lee K S, Beschorner W E, Vogel V G, Grochow L B, Braine H G, et al. Venoocclusive disease of the liver following bone marrow transplantation. Transplantation. 1987; 44 (6): 778-83.
14. Chao N. How I treat sinusoidal obstruction syndrome. Blood. 2014; 123 (26): 4023-6.
15. Corbacioglu S, Carreras E, Ansari M, Balduzzi A, Cesaro S, Dalle J H, et al. Diagnosis and severity criteria for sinusoidal obstruction syndrome/veno-occlusive disease in pediatric patients: a new classification from the European society for blood and marrow transplantation. Bone Marrow Transplant. 2018; 53 (2): 138-45.
16. Cairo M S, Cooke K R, Lazarus H M, and Chao N. Modified diagnostic criteria, grading classification and newly elucidated pathophysiology of hepatic SOS/VOD after haematopoietic cell transplantation. Br J Haematol. 2020; 190 (6): 822-36.
17. Dalle J H, and Giralt S A. Hepatic Veno-Occlusive Disease after Hematopoietic Stem Cell Transplantation: Risk Factors and Stratification, Prophylaxis, and Treatment. Biol Blood Marrow Transplant. 2015.
18. Mohty M, Malard F, Abecassis M, Aerts E, Alaskar A S, Aljurf M, et al. Sinusoidal obstruction syndrome/veno-occlusive disease: current situation and perspectives-a position statement from the European Society for Blood and Marrow Transplantation (EBMT). Bone Marrow Transplant. 2015; 50 (6): 781-9.
19. Group. F-NBW. Silver Spring (MD): Food and Drug Administration (US); 2016-.
20. Paczesny S, Hakim F T, Pidala J, Cooke K, Lathrop J, Griffith L M, et al. National Institutes of Health Consensus Development Project on Criteria for Clinical Trials in Chronic Graft-versus-Host Disease: III. The 2014 Biomarker Working Group Report. Biol Blood Marrow Transplant. 2015.
21. Bidgoli A, DePriest B P, Saatloo M V, Jiang H, Fu D, and Paczesny S. Current Definitions and Clinical Implications of Biomarkers in Graft-versus-Host Disease. Transplant Cell Ther. 2022.
22. Akil A, Zhang Q, Mumaw C L, Raiker N, Yu J, Velez de Mendizabal N, et al. Biomarkers for Diagnosis and Prognosis of Sinusoidal Obstruction Syndrome after Hematopoietic Cell Transplantation. Biol Blood Marrow Transplant. 2015;21 (10): 1739-45.
23. Jiang S, Penack O, Terzer T, Schult D, Majer-Lauterbach J, Radujkovic A, et al. Predicting sinusoidal obstruction syndrome after allogeneic stem cell transplantation with the EASIX biomarker panel. Haematologica. 2021; 106 (2): 446-53.
24. Cutler C, Kim H T, Ayanian S, Bradwin G, Revta C, Aldridge J, et al. Prediction of veno-occlusive disease using biomarkers of endothelial injury. Biol Blood Marrow Transplant. 2010; 16 (8): 1180-5.
25. Dvorak C C, Higham C, and Shimano K A. Transplant-Associated Thrombotic Microangiopathy in Pediatric Hematopoietic Cell Transplant Recipients: A Practical Approach to Diagnosis and Management. Front Pediatr. 2019; 7:133.
26. Vander Lugt M T, Braun T M, Hanash S, Ritz J, Ho V T, Antin J H, et al. ST2 as a marker for risk of therapy-resistant graft-versus-host disease and death. N Engl J Med. 2013;369 (6): 529-39.
27. Hartwell M J, Ozbek U, Holler E, Renteria A S, Major-Monfried H, Reddy P, et al. An early-biomarker algorithm predicts lethal graft-versus-host disease and survival. JCI insight. 2017;2 (3): e89798.
28. Rowan C M, Pike F, Cooke K R, Krance R, Carpenter P A, Duncan C, et al. Assessment of ST2 for risk of death following graft-versus-host disease in pediatric and adult age groups. Blood. 2020; 135 (17): 1428-37.
29. Brinkmann C R, Jensen L, Dagnaes-Hansen F, Holm I E, Endo Y, Fujita T, et al. Mitochondria and the lectin pathway of complement. The Journal of biological chemistry. 2013;288 (12): 8016-27.
30. Ding Q, Shen Y, Li D, Yang J, Yu J, Yin Z, et al. Ficolin-2 triggers antitumor effect by activating macrophages and CD8(+) T cells. Clin Immunol. 2017; 183:145-57.
31. van den Broek M A, Vreuls C P, Winstanley A, Jansen R L, van Bijnen A A, Dello S A, et al. Hyaluronic acid as a marker of hepatic sinusoidal obstruction syndrome secondary to oxaliplatin-based chemotherapy in patients with colorectal liver metastases. Ann Surg Oncol. 2013;20 (5): 1462-9.
32. Griesenauer B, and Paczesny S. The ST2/IL-33 Axis in Immune Cells during Inflammatory Diseases. Front Immunol. 2017; 8:475.
33. Zhang J, Ramadan A M, Griesenauer B, Li W, Turner M J, Liu C, et al. ST2 blockade reduces sST2-producing T cells while maintaining protective mST2-expressing T cells during graft-versus-host disease. Sci Transl Med. 2015;7 (308): 308ra160.
34. Nauffal M, Kim H T, Richardson P G, Soiffer R J, Antin J H, Cutler C, et al. Defibrotide: real-world management of veno-occlusive disease/sinusoidal obstructive syndrome after stem cell transplant. Blood Adv. 2022;6 (1): 181-8.
35. Bognar T, Bartelink I H, Egberts T C G, Rademaker C M A, Versluys A B, Slatter M A, et al. Association Between the Magnitude of Intravenous Busulfan Exposure and Development of Hepatic Veno-Occlusive Disease in Children and Young Adults Undergoing Myeloablative Allogeneic Hematopoietic Cell Transplantation. Transplant Cell Ther. 2022;28 (4): 196-202.
36. Strouse C, Zhang Y, Zhang M J, DiGilio A, Pasquini M, Horowitz M M, et al. Risk Score for the Development of Veno-Occlusive Disease after Allogeneic Hematopoietic Cell Transplant. Biol Blood Marrow Transplant. 2018;24 (10): 2072-80.
37. Higham C S, Shimano K A, Melton A, Kharbanda S, Chu J, Dara J, et al. A pilot trial of prophylactic defibrotide to prevent serious thrombotic microangiopathy in high-risk pediatric patients. Pediatr Blood Cancer. 2022;69 (5): e29641.
38. Mohty M. Malard F. Abecassis M. Aerts E. Alaskar A S. Aljurf M. et al. Revised diagnosis and severity criteria for sinusoidal obstruction syndrome/veno-occlusive disease in adult patients: a new classification from the European Society for Blood and Marrow Transplantation. Bone Marrow Transplant. 2016; 51 (7): 906-12.

1. A method of treating and/or preventing sinusoidal obstructive syndrome (SOS) in a subject receiving hematopoietic stem cell transplantation (HCT), said the method comprising:
(a) obtaining a biological sample from a subject receiving HCT;
(b) measuring in said biological sample from the subject the expression of L-Ficolin, hyaluronic acid (HA), and IL-1RL1 (ST2) by contacting the biological sample obtained from the subject with specific binding agents that specifically bind to each of the respective the biomarkers, wherein each specific binding agent forms a complex with the respective biomarker;
(c) detecting the agent-biomarker complexes, thereby determining the biomarker expression level, wherein decreased expression level of L-ficolin, elevated expression level of HA, and/or elevated expression level of ST2 as compared to a control identifies a subject at risk of SOS; and
(d) treating said patient at risk of SOS with a therapeutic agent.

2. The method of claim 1, wherein the therapeutic agent is defibrotide, eculizumab, narsoplimab, n-acetyl-1-cysteine (NAC), and/or recombinant human soluble thrombomodulin alpha (rhTM).

3. (canceled)

4. The method of claim 1, wherein the biological sample is blood, serum, plasma, urine, spinal fluid, saliva, lacrimal fluid, or sweat.

5. (canceled)

6. (canceled)

7. The method of claim 1, wherein detecting is further defined as performing an enzyme-linked immunosorbent assay (ELISA).

8. The method of claim 1, wherein the sample was obtained from the subject three days post-HCT.

9. The method of claim 1, wherein the sample was obtained from the subject between 1 and 30 days post-HCT.

10. The method of claim 1, wherein the sample was obtained from the subject between 2 and 15 days post-HCT.

11. The method of claim 1, wherein the sample was obtained from the subject between three and seven days post-HCT.

12. The method of claim 1, wherein the sample was obtained from the subject at three days post-HCT.

13. The method of claim 1, wherein the subject is less than 18 years of age.

14. The method of claim 1, wherein the subject is less than 5 years of age.

15. (canceled)

16. The method of claim 1, wherein the control is a healthy subject.

17. The method of claim 1, wherein an expression level of L-ficolin less than 1100 ng/mL, an expression level of HA greater than 200 ng/mL, and/or an expression level of ST2 greater than 45 ng/mL identifies a subject at risk of SOS.

18. The method of claim 1, wherein decreased expression level of L-ficolin and elevated expression level of ST2 as compared to a control identifies a subject at risk of SOS.

19. The method of claim 1, wherein decreased expression level of L-ficolin, elevated expression level of HA, and elevated expression level of ST2 as compared to a control identifies a subject at risk of SOS.

20. The method of claim 3, wherein administering defibrotide results in increased expression levels of L-ficolin, decreased expression level of HA, and/or decreased expressed level of ST2 as compared to expression levels prior to defibrotide treatment.

21. (canceled)

22. The method of claim 3, wherein defibrotide is administered for less than 21 days.

23. The method of claim 1, wherein the AUC of each of the biomarkers is greater than 0.5.

24-27. (canceled)

28. The method of claim 1, wherein an expression level of L-ficolin less than 1100 ng/mL, an expression level of HA greater than 200 ng/mL, and/or an expression level of ST2 greater than 45 ng/mL indicates decreased survival as compared to median survival in a subject having SOS.

29. An in vitro method for measuring the expression of the antigens L-ficolin, HA, and ST2 comprising:
(a) obtaining a sample from a subject having received a hematopoietic stem cell transplantation (HCT);
(b) contacting said sample with an anti-L-ficolin antibody, an anti-HA antibody, and an anti-ST2 antibody to form antigen-antibody complexes; and
(c) detecting the antigen-antibody complexes using detectable moieties that distinctly bind each of the antibodies, thereby measuring the expression of the antigens L-ficolin, HA, and ST2 in said sample.

30-49. (canceled)
